(19) United States
(12) Patent Application Publication
(10) Pub. No.: US 2004/0167330 A1
(43) Pub. Date: Aug. 26, 2004

(54) Novel chelating agents and conjugates thereof, their synthesis and use as diagnois and therapeutic agents

(76) Inventors: Ivan Lukes, Prague (CZ); Petr Hermann, Prague (CZ)

Correspondence Address: HENRY M FEIEREISEN, LLC, 350 FIFTH AVENUE, SUITE 4714, NEW YORK, NY, 10118, US

(21) Appl. No.: 10/484,391

(22) PCT Filed: Jul. 16, 2002
(86) PCT No.: PCT/EP02/07920

(51) Int. Cl. C07D 257/02

(52) U.S. Cl. 540/474

(57) ABSTRACT

The present invention relates to novel bifunctional chelates that are based on asymmetrical cyclen derivatives. The chelates contain either three acetates and one methylphosphonic arm or three acetates and one methylphosphonic arm enabling to link the chelate through P-alkyl within phosphoric acid derivative or through P—O-alkyl within phosphonic derivative to any organic back-bone suited for targeting. Suitable targeting moieties are monoclonal antibodies, their fragments and recombinant derivatives such as single chain antibodies, diabodies, triabodies, humanized, human or chimeric variants but also peptides, aptamers, spiegelmers, nucleotides, anti sense oligomers and conventional small molecules. These novel bifunctional chelates are suited for the production of kits for the routine labelling of targeting moieties to be used in radiotherapy with radiometals such as Yttrium-90, or for Magnetic Resonance Imagining (MRI) using Gadolinium.

### FIELD OF THE INVENTION

The present invention relates to novel bifunctional chelates that are based on asymmetrical cyclen derivatives. The chelates contain either three acetates and one methylphosphinic arm or three acetates and one methylphosphonic arm enabling to link the chelate through P-alkyl within phosphinic acid derivative or through P—O-alkyl within phosphonic derivative to any organic backbone suited for targeting. Suitable targeting moieties are monoclonal antibodies, their fragments and recombinant derivatives such as single chain antibodies, diabodies, triabodies, humanized, human or chimeric variants but also peptides, aptamers, spiegelmers, nucleotides, anti sense oligomers and conventional small molecules. These novel bifunctional chelates are suited for the production of kits for the routine labelling of targeting moieties to be used in radiotherapy with radiometals such as Yttrium-90, or for Magnetic Resonance Imaging (MRI) using Gadolinium.

### BACKGROUND OF THE INVENTION

Polydentate ligands, such as DTPA (diethylenetriaminepentaacetic acid), macrocyclic TETA (1,4,8,11-tetraazacyclotetradecane-1,4,8,11-tetraacetic acid), and DOTA (1,4,7,10-tetraazacyclododecane-1,4,7,10-tetraacetic acid) form thermodynamically and kinetically very stable metal chelate complexes even with labile metal ions as the first-row transition-metal divalent ions or trivalent lanthanides (Lindoy L. F.: Adv. Inorg. Chem. 1998, 45, 75; Wainwright K. P.: Coord. Chem. Rev. 1997, 166, 35; Lincoln S. F.: Coord. Chem. Rev. 1997, 166, 255; Meyer M., Dahaoui-Gindrey V., Lecomte C., Guilard R.: Coord. Chem. Rev. 1998, 178-180, 1313; Hancock R. D., Maumela H., de Sousa A. S.: Coord. Chem. Rev. 1996, 148, 315). The properties of macrocyclic ligands have been elaborated while designing both Gd³⁺ based magnetic resonance imaging (MRI) contrast agents (Parker D. in: Comprehensive Supramolecular Chemistry (Lehn J.-M., Ed.), Vol. 10, pp. 487-536. Pergamon Press, Oxford 1996; Aime S., Botta M., Fasano M., Terreno E.: Chem. Soc. Rev. 1998, 27, 19; Caravan P., Ellison J. J., Mc Murry T. J., Laufer R. B.: Chem. Rev. 1999, 99, 2293; Aime S., Botta M., Fasano M., Terreno E.: Acc. Chem. Res. 1999, 32, 941; Botta M.: Eur. J. Inorg. Chem. 2000, 399) as well as diagnostic and/or therapeutic radiopharmaceuticals based on metal radionuclides (Anderson C. J., Welch M. J.: Chem. Rev. 1999, 99, 2219; Volkert W. A., Hoffmann T. J.: Chem. Rev. 1999, 99, 2269; Reichert D. E., Lewis J. S., Anderson C. J.: Coord. Chem. Rev. 1999, 184, 3; Liu S., Edwards D. S.: Biocojugate Chem. 2601, 12, 7). For radiopharmaceutical use, targeting moieties have to be linked to the radio metal chelate complex. The chelate is called bifunctional due to its ability to bind to the targeting moiety on one hand and to complex the radiometal on the other hand.

Targeting moieties such as monoclonal antibodies (Mabs) were described by Koehler and Milstein in mid seventies (Koehler G. and Milstein C.: Nature 1975, 256, 495-497). Since then, investigators tried to develop these proteins of unprecedented specificity as diagnostics and therapeutics. Success in the diagnostic area was achieved very fast, but only recently, despite of significant efforts of many research groups, the first therapeutically successful Mabs were approved by FDA and EMEA to treat cancer. So far, the Mabs approved for the therapy of cancer, are recombinantly manipulated chimeric or humanized Mabs inducing their therapeutic effects by interfering with cell surface receptor function (erb B2 receptor: Herceptin) or mediating ADCC and CDC via an appropriate Fc moiety (CD 20: ROCHE-Rituxan).

More recently, comparative clinical studies showed that Ibritumomab, a mouse MAb selective for CD20 (IDEC-Y2B8) and labelled with Yttrium-90 (Y-90), is more efficacious with respect to clinical efficacy for the treatment of non Hodgkin's lymphoma than its chimeric unlabelled but cytotoxic recombinant variant MAb Rituximab (ROCHE-Rituxan). The increased therapeutic efficacy of Ibritumomab-tiuxetan (Zevalin) can be explained with the bystander effect which is caused by the pure β-emitting, high energy (2.3 MeV) radionucleotide ⁹⁰Y, allowing irradiation of CD 20 negative lymphoma cells within a range of 9 mm apart from CD 20 positive tumor cells.

In the case of Ibritumomab, Y-90 is relatively stable attached to the Mab Y2B8 via a covalently bound chelator-linker called tiuxetan (MX-DTPA) (Brechbiel M. W. et. al.: Inorg. Chem. 1986, 25, 2772; Cummins et al.: Bioconjugate Chem. 1991, 2, 180; Brechbiel M. W. and Gansow O. A.: Bioconjugate Chem. 1991, 2, 187). To increase the stability of the chelating group for Y-90, Quadri and Mohammadpour (Bioorg. Med. Chem. Lett. 1992, 2, 1661-1664) synthesised benzyl-methyl-DTPA chelates carrying the benzyl in the C2 and the methyl in the C3 position. However these so called ITC-2B3M-DTPA reagents did not show an increased chelating stability for Y-90 in comparison to ITC MX-DTPA. Nevertheless, these chelates are used in experimental studies intended to treat ovarian cancers after intraperitoneal injection (Borchardt et al.: J. Nucl. Med. 1998, 39, 476-484).

The most stable chelates for Y-90 or In-111 are the DOTAs which are attached to a Mab using different linker chemistries (Li M. and Meares C. F.: Bioconjugate Chem. 1993, 4, 275-283). However, the major drawback limiting the use of DOTA chelates are the physicochemical conditions which need to be applied for the incorporation of the radiometal in the Mab-DOTA immunoconjugate (Lewis et al.: Bioconjugate Chem. 1994, 5, 565-576). Mab-DOTA immunoconjugates have to be incubated at elevated temperatures for a long period of time damaging the Mab component of the immunoconjugate and making the radiolabelling procedure inappropriate for routine use.

In addition, a damage of the Mab moiety can be detected by a significant reduction of the immunoreactive fraction of the immunoconjugate resulting in an increased unfavourable liver accumulation compared to immunoconjugates having immunoreactivities >90% (German patent application: 100 16 877.9). Some investigators tried to reduce the issue of liver accumulation by the introduction of enzymatically cleavable peptide linkers between the DOTA and the Mab moiety (Peterson J. J. and Meares C.: Bioconjugate Chem. 1999, 10, 553-557). These linkers eventually allow a faster elimination of the DOTA-chelate from the liver following cleavage by lysosomal enzymes such as catepsin B or D. However, enzymatically cleavable chelates are not only cleaved in liver tissues but in all tissues in which the Mab-linker-DOTA chelate gets internalized and processed via the lysosomal compartment. This can happen in the target tissues, such as tumors, unfavorably reducing the radiation dose to the target tissue.

In search for other ligands with similar or better properties, especially for the faster complexation than common acetate derivatives, research has also been focused on synthesis and investigation of azamacrocycles with four phosphonic or phosphinic acid pendant arms. Complexes with the phosphorus ligands exhibit higher selectivity in complexation and sufficient thermodynamic stability (Sherry A. D.: J. Alloys Compd. 1997, 249, 153; Belskii F. I., Polikarpov Yu. M., Kabachnik M. I.: Usp. Khim. 1992, 61, 415; Rohovec J., Kyvala M., Vojtí\check{s}ek P., Hermann P., Lukeś I.: Eur. J. Inorg. Chem. 2000, 195; Bazakas K., Luke\check{s} I.: J. Chem. Soc., Dalton Trans. 1995, 1133). A comparison of the complexing properties of cyclen and cyclam derivatives containing acetic acid pendant arms on one hand and their methylphosphonic or methylphosphinic acid analogues on the other has been summarised and published recently (Luke\check{s} I., Kotek J., Vojtí\check{s}ek P., Hermann P.: Coord. Chem. Rev. 2001, 216-217, 287).

### DESCRIPTION OF THE INVENTION

To our surprise, cyclic compounds having three carboxylic acid arms and one phosphinic or phosphonic acid arm showed advantageous and unexpected characteristics with respect to metal chelate complex stability and metal incorporation. The chelates preferably contain either three acetates or their optionally substituted amides and one methylphosphonic arm (phosphonic derivative) or three acetates or their optionally substituted amides and one methylphosphinic arm (phosphinic derivative) or three acetates or their optionally substituted amides and one methylphosphine oxide arm (phosphine oxide derivative).

Thus, the present invention relates to a compound of formula I

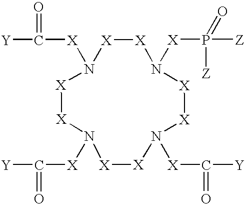

wherein

each X is independently selected from C(R¹)₂ or CR¹R²,

each Z is independently OH, R¹, R², OR¹, OR² or OM and M is a cation,

Y is independently OH, OM, OR¹, OR², NR¹R², N(R¹)₂ or N(R²)₂ and M is a cation,

each R¹ is independently selected from H or an organic radical having from 1-20 carbon atoms, and

each R² is independently selected from H, a functional group or an organic radical having from 1-20 carbon atoms carrying at least one functional group,

or an optical isomer, a coordination compound or a salt thereof.

In a preferred embodiment of the invention each X is CH₂. It should be noted, however, that in some cases it may be preferred that one group X has the meaning CHR¹ or CHR², wherein R¹ and R² is different from H.

The term “organic radical having from 1-20 carbon atoms” according to the present invention particularly relates to C₁-C₁₀ alkyl, C₂-C₁₀ alkenyl, C₂-C₂₀ alkynyl, C₃-C₈ cycloalkyl, C₅-C₁₀(hetero)aryl radicals including aryl or cycloalkyl radicals containing further substituents such as alkyl groups. Further, the R¹ radicals may contain heteroatoms such as F, Br, Cl, F, O, N, S and/or P.

The symbol R² is defined like R¹ but additionally may be or contain a functional group, particularly a group which is suitable for conjugating the compound of formula I to a binding partner such as a biomolecule. Numerous examples of such coupling groups which e.g. are capable of selectively reacting with amino, thio or hydroxy groups of biomolecules are known in the art. Specific examples for functional groups are OR¹, Cl, Br, I, NO₂, N(R¹)₂, COOR¹, NCS and NHCOCH₂Br, wherein R¹ is defined as described above.

The substituent Z on the phosphorous atom may be bound thereto via a carbon atom or an oxygen atom. When the binding is via a carbon atom the compound of formula I is a phosphinic acid derivative. When the binding occurs via an oxygen atom the compound of formula I is a phosphonic acid derivative. The conjugation to binding partners preferably occurs via the substituent Z.

Examples of Z are H, OH, O—C₁-C₄ alkyl such as OC₂H₅, C₁₋₄ alkyl such as CH₃, —Oₙ-alkaryl such as —CH₂ phenyl, —CH₂C₆H₄NO₂ or —CH₂C₆H₄NH₂, —Oₙ, C₁-C₄ hydroxy alkyl such as CH₂OH, —Oₙ—C₁-C₄ alkyl carboxyl such as CH₂CO₂H or —Oₙ—C₁-C₄ amino alkyl such as CH₂NH₂, wherein n is 0 or 1, or OM, wherein M is a metal cation. More preferably Z contains a functional group capable of coupling to a binding partner, e.g. a biomolecule.

Particularly preferred meanings of Z are —Oₙ—(CH₂)₁₋₆-Q, —Oₙ—(CH₂)₁₋₄—Ph-Q or —Oₙ—Ph-Q, wherein Q is —NH₂, —COOH, —NCS or —NHCOCH₂Br and n is 0 or 1.

The substituent Y may be H, or OM, wherein M is a cation, e.g. an alkaline metal cation, an alkaline earth metal cation or an organic cation such as an amine cation, e.g. a quaternary ammonium ion. The carboxylic acids arms, however, may also be derivatized, e.g. as an ester, an amide or the like.

The compounds of the present invention may be complexed with metal ions, preferably with metal ions in the oxidation state ≧+2. Suitable examples of metal ions are transition metals, lantamides, actinides, but also main group metal ions. In a preferred embodiment the metal is a radioisotope, e.g. ⁶⁴Cu, ⁶⁷Cu, ⁶⁷Ga, ⁹⁰Y, ¹¹¹In, ¹⁵³Sm, ¹⁶⁶Ho, ¹⁷⁷Lu, ²⁰¹Tl, ²¹²Bi and combinations thereof. In a further preferred embodiment the metal is Gd.

The compound or the metal complex of the invention may be coupled to a binding partner, particularly a biomolecule such as a peptide, a protein, a glycoprotein, an oligo- or polysaccharide, an oligo- and polyaminosugar or a nucleic acid. Most preferably the biomolecule is an antibody, e.g. a monoclonal antibody, a chimerized antibody, a humanized antibody, a recombinant antibody, e.g. a single chain antibody or an antibody fragment which may be obtained by proteolysis from a complete antibody or by genetic manipulation of antibody-encoding nucleic acids. Methods for preparing suitable antibodies or antibody fragments are known to the skilled person.

Formula 1 represents preferred embodiments of compounds, namely monophosphonic DO3A-P and monophosphinic DO3A-P^{R} acid analogues of DOTA.

Complexes of the chelates of compounds of formula (I) exhibit the following unexpected properties:

1) Complexation kinetics of DO3A-P and DO3A-P^{R} and their derivatives are faster than the carboxylic ones. Kinetic and thermodynamic stability of the complexes are high, similar to that observed for DOTA complexes. Thus, covalent conjugates consisting of a targeting moiety and a chelate (furtheron called immuno conjugate) allow both a fast incorporation of the radiometal at physiological temperature as well as avoid any loss of radiometal from the chelate in vivo.

2) Both phosphinic and phosphonic acid groups enable the coupling of a chelate through P-alkyl within the phosphinic acid derivative or phosphine oxide derivative or through P—O-alkyl within the phosphonic derivative to the targeting moiety. Formation of the P-alkyl and P—O-alkyl linkers do not influence coordination ability of the phosphorus group, in contrast to derivatives of DOTA monoamide.

3) In contrast to DOTA, DO3A-P and DO3A-P^{R} and the corresponding phosphine oxide derivative are more specific for hard ions such as lanthanides.

4) Both DO3A-P and DO3A-P^{R} and the corresponding phosphine oxide derivative have the advantageous property to coordinate one water molecule being crucial in magnetic resonance e.g. MRI applications. Due to the size of phosphonic/phosphinic/phosphine oxide groups, the water molecule is exchanged much faster and the respective contrast agents (phosphinic or phosphonic derivatives based on Gd) are more efficient.

The compounds of formula (I) may be synthesized by a protocol comprising a Mannich reaction between DO3A derivatives and phosphorus acid derivatives containing a P—H bond.

>N—H+CH₂O+H—PZ(O)(OR¹)→>N—CH₂—PZ(O)(OR¹)

>N—H+CH₂O+H—P(O)(OR¹)(OZ)→>N—CH₂—P(O)(OR¹)(OZ)

The DO3A as well as any amine, which is not sterically hindered, reacts with phosphorus components such as phosphinic acids or their esters (H—PZ(O)(OR¹)) and phosphorous acid or its monoesters or its diesters (H—P(O)(OR¹)(OZ)) and formaldehyde or paraformaldehyde. The reaction may be performed in a non-aqueous medium, usually with esters, in solvents such as as benzene, toluene or THF. Formaldehyde is preferably introduced as paraformaldehyde (excess 200-400%). In aqueous medium, the reaction may be carried out with water-soluble phosphorus components. Formaldehyde is preferably used in form of saturated aqueous solution as paraformaldehyde and in excess (200-400%). In addition to water, a HCl solution from very low concentration to azeotropic HCl may also be used at a temperature range from 40° C. up to reflux temperature. Products from reactions in non-aqueous solutions with phosphorus ester derivatives may have to be purified by column chromatography e.g. on SiO₂ or alumina. Usually, reactions in an aqueous solution give higher yields. Products can be purified by chromatography on ion exchange resins.

Further, the compounds may be prepared by a Mannich reaction, e.g. in an alkaline solution at pH 8-10 in methanol with dimethylphosphate and methylesters of phosphinic acids or in ethanol with the corresponding ethylesters. A preferred general procedure comprises reacting a secondary amine, phosphorous acid methylester (3-20 equivalents) and aqueous formaldehyde (30%, 3-20 equivalents) in methanol at about pH 9 (adjusted by addition of a tertiary amine, e.g. diisopropylethylamine or another sterically hindered amine) in a closed flask under suitable conditions, e.g. at 70-90° C. for 10-48 h. Then, the reaction mixture is cooled and evaporated. The reaction product may be purified on Al₂O₃, SiO₂ or ion-exchange resins.

For the formation of immunoconjugates, a reactive functional group is introduced into the compound. The resulting novel bifunctional chelating agents have isothiocyanate or other functional groups preferably on the phosphorus arm allowing smooth reaction with OH, NH₂ or SH groups of the targeting moiety.

The novel bifunctional chelating agents are particularly suitable for complexation of lanthanides and yttrium. For complexation, oxides or common salts such as nitrates, chlorides or acetates of metals such as lanthanides and yttrium can be used. The ions may be incorporated in the chelates at ambient temperature and about neutral pH. The process of complexation starts at approximately pH 5 and is slowly increased after 10 minutes to approximately pH 7. Under these conditions the complexation is finished within 30 minutes, as shown using NMR.

Further, the present invention relates to a pharmaceutical composition comprising a compound, a metal complex or a conjugate as described above together with pharmaceutically acceptable carriers, diluents or adjuvants. The composition may be suitable for diagnostic applications such as radioimaging or magnetic resonance imaging. On the other hand, the composition may be suitable for therapeutic applications such as radiotherapy or neutron capture therapy.

In addition to the use in nuclear medicine, the presently available gadolinium(III) based MRI contrast agents do not meet the theoretical value of relaxivity and, therefore more efficient contrast agents are highly desired. Relaxivity can be improved either by increasing the water exchange rate or by covalent/non-covalent binding to a large molecule and thus, the novel Gd(III) complexes using the novel bifunctional chelates described above can be linked to an organic backbone of e.g. aminosugars or proteins. In an especially preferred embodiment, the complexes may be coupled non-covalently, e.g. via hydrophobic side chains to biomolecules, such as human serum albumin. The efficiency of these high-molecular weight aggregates used as contrast agents in MRI is higher than that of the isolated complexes. Particularly, non-covalent conjugates have a longer half-life in blood and consequently slower pharmacokinetics.

The composition is preferably an injectible liquid. It should be noted, however, that other forms of administration and formulations are possible. In this context it is referred to known administration protocols for metal chelate complexes, particularly metal chelate complexes conjugated to biomolecules such as polypeptides, peptides, saccharides and/or nucleic acids.

Finally, the present invention relates to a method of administering a subject in need thereof a diagnostically or therapeutically effective amount of a compound, a metal complex or a conjugate as described above together With pharmaceutically acceptable carriers, diluents or adjuvants.

### EXAMPLES

Synthesis of Bifunctional Chelates

If not otherwise stated commercial chemicals were used in the syntheses.

### Example 1

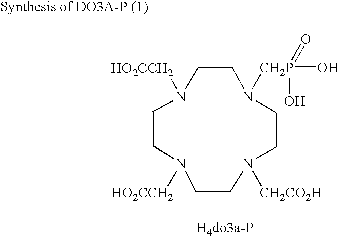

1 g DO3A (2.89 mmol) and 1.85 ml HP(O)(OEt)₂ (14.4 mmol, 5 equiv.) was dissolved in 5 ml of HCl (1:1) in 25 ml flask equipped with reflux condenser. The flask was flushed with argon. At 80° C., 0.52 g (CH₂O)ₙ (17.3 mmol, 6 equiv.) was slowly added into the flask over 5 h. The reaction mixture was heated under gentle reflux for 50 h. Solvents were removed on rotary evaporator, the residue was dissolved in 2 ml of water, decolorized with charcoal (stirring a day at 60° C.). Charcoal was filtred off and the filtrate concentrated and applied onto Dowex 50 column (100 ml, H⁺-form). Non-aminic impurities were eluated with water (200 ml) and cyclic compounds were eluated by 5% aq. ammonia. Fractions containing amines were evaporated in vacuo and the residue was dissolved in 2 ml of water. The solution was applied onto Amberlite 50CG column (100 ml) and the column was eluated with water. The first several 40 ml fractions contained pure product, later fractions contain unreacted DO3A and some unidentified impurities. Fractions containing pure ligand were evaporated. The residue was dissolved in 5 ml of water and briefly heated with charcoal, filtered and solution was evaporated again. The residue was dissolved in 1 ml of water. The solution was slowly dropped into vigorously stirred EtOH (250 ml). It was left overnight, centrifugated, washed with EtOH and dried at 50° C. for several hours in vacuo. The white solid was left to equilibrate with air moisture overnight. Yield 1.15 g (81%) of DO3A-P.3H₂O. The compound was analysed using NMR.

³¹P NMR (1 M NaOD/D₂O): 10.0 ppm

¹H NMR (1 M NaOD/D₂O): 3.05 ppm (d, 2H, ²J(PH)=10.8 Hz, CH₂—P), 2.81-3.37 ppm (m, 16H, ring CH₂), 3.30 ppm (s, 2H, CH₂—COOH), 3.37 ppm (s, 4H, CH₂—COOH).

¹³C NMR (1 M NaOD/D₂O): 51.72-53.54 ppm (ring C), 52.6 ppm (¹J(PC)=130.2 Hz, C—P), 59.53 and 59.57 ppm (acetic CH₂), 179.14 and 179.98 ppm (COOH).

ESI/MS (positive): 441.5 (M+H⁺), 463.2 (M+Na⁺)

Elementary analysis (calc.): C 36.20 (36.44) H 6.80 (7.13) N 11.48 (11.33)

### Example 2

Synthesis of DO3A-P (1)

The same procedure as in Example 1 was used except that phosphorus acid (1.18 g) was used instead of the diethyl ester. Yield of trihydrate of 1 was 0.95 g

### Example 3

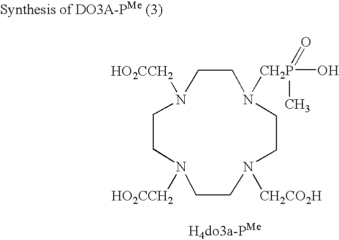

a) Synthesis of MePO₂H₂ (2) (Performed Following the Procedure Published by K. Issleib et all. Z. Anorg. Alig. Chem. 1985, 530, 16 and E. A. Boyd et all Tetrahedron Lett. 1994, 35, 4223)

A fine suspension of 20.8 g (0.25 mol) of dried NH₄H₂PO₂ in 120 ml of hexamethyldisilazane was refluxed with stirring for 6 h (ammonia formed was ventilated out) in argon atmosphere resulting in a solution of intermediate HP(OSiMe₃)₂. After cooling to 0° C., 200 ml of dry CH₂Cl₂ was added. Solution of MeI (15.6 ml, 0.25 mol) in 50 ml of dry CH₂Cl₂ was slowly dropped into the phosphine solution with cooling (0° C.) and stirring. The reaction mixture was stirred overnight at room temperature. Methanol (20 ml) was added with cooling and, after 30 min, the solution was filtered. Volatiles were removed using a rotavapor leaving an oil pure enough for the next step. The compound was analysed using NMR.

b) Esterification of MePO₂H₂ (Based on the Procedure Published by Y. R. Dumond et al., Org. Lett. 2000, 2, 3341)

Acid 2 (10 g, 0.125 mol) from the previous example was dissolved in 100 ml THF and 20 ml of Si(OMe)₄ or Si(OEt)₄ was slowly added dropwise. The mixture was refluxed overnight and volatiles were removed using a rotavapor. The residue was partitioned between acetonitrile and hexane. The acetonitrile layer was decanted, the solvent was moved using the rotavapor and the residue was distilled on a short column. Yield of MeP(O)(H)(OMe) was 75% (b.p. 65-69° C./15 torr) and MeP(O)(H)OEt) was 81% (b.p. 83-87° C./15 torr). The compound was analysed using NMR.

Ethylester: ³¹P NMR (CDCl₃): 32.8 ppm (¹J(PH)=545 Hz)

Methylester: ³¹P NMR (CDCl₃): 31.3 ppm (¹J(PH)=555 Hz)

c) Reaction of DO3A with MePO₂H₂ (2). Formation of DO3A-P^{Me} (3)

1 g DO3A (2.3 mmol) and 0.52 g MePO₂H₂ (2) (10 mmol, 4.5 equiv.) were dissolved in 10 ml of azeotropic HCl in a 50 ml flask equipped with the reflux condenser. The solution was bubbled with argon for 10 min. Under argon, 0.5 ml of aqueous CH₂O (36%, 3 equiv.) was added into the flask. The reaction mixture was heated at gentle reflux temperature for 24 h. Additionally, 0.5 ml of aqueous CH₂O (36%, 3 equiv.) was added and the mixture was refluxed for another 6 h. Solvents were removed using a rotary evaporator (inert atmosphere is not necessary), the residue was dissolved in 2 ml of water, decolorized with charcoal and applied onto a Dowex 50 column (100 ml, H⁺-form). Non-aminic impurities were eluted with water (200 ml) and cyclic compounds were eluted by 5% aqueous ammonia. Fractions containing amines were evaporated in vacuo and the residue was dissolved in 2 ml of water. The solution was applied onto Amberlite 50CG column (100 ml) and the column was eluted with water. The first two 100 ml fractions contained pure product, fractions 4 and 5 contained the pure inner lactam (16). Fractions containing pure compounds were evaporated and dissolved in 1 ml of conc. HCl. THF (50 ml) was slowly (3 h) dropped into the solutions with stirring. The solids were filtered, washed with THF and dried in vacuo over P₂O₅. Yield was 0.78 g of DO3A-P^{Me}.2HCl.3H₂O (3.2HCl₃H₂O) and 0.20 g DO3A-lactam.2HCl.2H₂O (4.2HCl.2H₂O).

The compound was analysed using NMR.

³¹P NMR (D₂O, 90° C.): 34.8 ppm;

¹H NMR (D₂O, 90° C.): 1.42 ppm (d, 3H, ²J(PH)=13 Hz), 3.12 ppm (d, 2H, ²J(PH)=7.9 Hz), 3.43 pm (s, 2H, CH₂—COOH), 3.58 ppm (s, 4H, CH₂—COOH), 3.08-3.40 (m, 16H, ring CH₂)

ESI/MS (positive: 439.6 (M+H⁺), 461.9 M+Na⁺)

Elementary analysis (calc.): C 32.9 (33.99) H 7.35 (6.95) N 9.32 (9.91) Cl 11.76 (12.54)

d) Reaction of DO3A with MeP(O)(H)(OEt). Formation of DO3A-P^{Me} (3)

A procedure similar to Example 1 c was used, except that instead of acid (2) its ethylester (1.13 g) was applied. The yield of hydrochloride hydrate of (3) was 0.73 g.

### Example 4

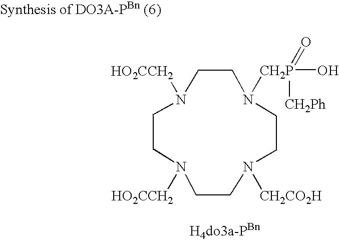

a) Synthesis of Benzylphosphinic Acid PhCH₂PO₂H₂ (5) and Its Methyl and Ethyl Esters

The acid was prepared as in compound (7) using 10.4 g (0.125 mol) of NH₄H₂PO₂ and benzyl bromide (10.8 g, 0.063 mol) instead of MeI and purified as follows. The oil after solvent removal was dissolved in water and precipitated Bn₂PO₂H(=(PhCH₂)₂PO₂H) was filtered off and washed with water. Water was removed in vacuo and the residue was chromatographed on Amberlite CG50 with water elution. Fractions containing pure BnPO₂H₂ were pooled and evaporation of water left crystalline PhCH₂PO₂H₂ in a yield of 54%. The compound was analysed using NMR.

³¹P NMR (CDCl₃): 35.9 ppm (¹J(PH)=560 Hz)

¹H NMR (CDCl₃): 3.12 ppm (dd, 2H, ²J(PH)=18.6 Hz, ³J(HH)=1.8 Hz); 6.96 ppm (dt, 1H, ¹J(PH)=561 Hz, ³J(HH)=1.8 Hz); 7.21-7.34 (m, 5H, aryl)

Esterification (methyl- and ethylester) of benzylphosphinic acid was carried out in the same way as esterification of methylphosphinic acid (2) and was distilled afterwards (ethylester at 110-115° C./0.025 torr).

b) Synthesis of benzylphosphinic Acid PhCH₂PO₂H₂ (5)

Ester P(OSiMe₃)(OEt)(CH(OEt)₂) (26.8 g, 0.1 mol) was dissolved in 100 ml of dry CH₂Cl₂. Benzylbromide (17.1 g, 0.1 mol) was dissolved in 100 ml of dry CH₂Cl₂ and slowly dropped into solution of silyl ester with stirring and cooling. It was left overnight at room temperature. MeOH (30 ml) was added and volatiles were removed using a rotavapor. The residue was dissolved in 25 ml of EtOH, 25 ml of conc. HCl was added and the solution was refluxed overnight. Solvents were evaporated in vacuo. The residue was dissolved in water, decolorised by charcoal and evaporated to dryness to give product in a yield of 91%.

c) Synthesis of benzylphosphinic Acid PhCH₂PO₂H₂ (5)

A solution of sodium salt of ester HP(O)(OEt)(CH(OEt)₂) (made from 9.81 g of the ester, 0.05 mol) was prepared starting from the ester solution in 30 ml of toluene by dropping NaOEt solution in 10 ml of dry EtOH (made equivalent amount of Na). Toluene (10 ml) solution of benzyl bromide (8.55 g, 0.05 mol) was dropped into sodium salt solution and the mixture was stirred for 20 h at room temperature. Solvent was removed using a rotavapor and protected ester was hydrolysed in refluxing aqueous HCl. After evaporation in vacuo, the benzylphosphinic acid was purified on Amberlite 50CG column with elution of water. Yield was 75%.

d) Reaction of DO3A with C₆H₅CH₂PO₂H₂ (5). Formation of DO3A-PBn (6)

0.65 g DO3A (1.5 mmol) and 0.9 g C₆H₅CH₂PO₂H₂ (5) (6.7 mmol, 4.5 equiv.) were dissolved in 10 ml of azeotropic HCl in a 50 ml flask equipped with the reflux condenser. The flask was flushed with argon. 0.5 ml of aqueous CH₂O (36%, 3 equiv.) was added into the flask. The reaction mixture was heated under gentle reflux for 24 h. Additionally, 0.5 ml of aqueous CH₂O (36%, 3 equiv.) was added and mixture was refluxed for another 30 h. Solvents were removed using a rotary evaporator, the residue was dissolved in 2 ml of water, decolourised with charcoal and applied onto Dowex 50 column (100 ml, H⁺-form). Non-aminic impurities were eluted with water (200 ml) and cyclic compounds were eluted by 5% aq. ammonia. Fractions containing amines were evaporated in vacuo and the residue was dissolved in 2 ml of water. The solution was applied onto Amberlite 50CG column (100 ml) and the column was eluted with water. The first two 100 ml fractions contained pure product, later fractions contained the inner lactam and unreacted H₃do3a. Fractions containing pure chelate were evaporated and the residue was dissolved in 1 ml of water. THF (50 ml) was slowly (3 h) dropped into the solutions with stirring. The solid was filtered, washed with THF and dried in vacuo over P₂O₅. Yield was 0.51 g of DO3A-P^{Bn} isolated as trihydrate. The compound was analysed.

Elementary analysis (calc.): C 45.91 (46.47) H 8.05 (7.27) N 9.06 (9.85).

³¹P NMR (KOD/D₂O, 90° C.): 37.9 ppm;

¹H NMR (KOD/D₂O, 90° C.): 2.55-2.86 ppm (m, 16H, ring CH₂), 2.975 ppm (d, 2H, ²J(PH)=1.2 Hz), 3.10 ppm (d, 2H, ²J(PH)=12 Hz), 3.15 pm (s, 2H, CH₂—COOH), 3.17 ppm (s, 4H, CH₂—COOH), 7.18-7.29 ppm (5H, aromatic ring);

¹³C NMR (KOD/D₂O, 90° C.): 43.33 ppm (d, ¹J(PC)=77 Hz); 53.81-54.86 ppm (azacycle carbons), 55.73 (d, ¹J(PC)=92 Hz), 61.80 ppm and 62.242 ppm (acetate carbons), 128.9-138.5 ppm (phenyl ring), 171.61 and 182.76 (carboxylate carbons)

ESI/MS (positive): 516.1 (M+H⁺), 527.7 (M+Na⁺)

### Example 5

Synthesis of DO3A-P^{Bn} (6)

A procedure similar to the one in Example 4 was used, except that instead of acid itself (4), its methylester (1.15 g) was applied. Yield of 5 was 0.73 g.

### Example 6

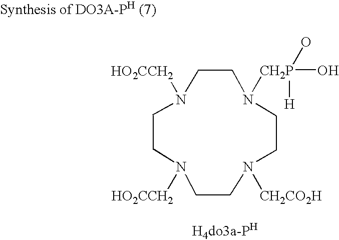

1 g DO3A (2.3 mmol) and 0.66 g H₃PO₂ (10 mmol, 4.5 equiv.) were dissolved in 10 ml water and 2 ml of conc. HCl using a 50 ml flask. The flask was closed with rubber septum and flushed with argon. 0.82 g of aqueous CH₂O (36%, 4.5 equiv.) was added into the flask. The reaction mixture was heated at 80° C. for 20 h. Solvents were removed using a rotary evaporator, the residue was dissolved in 2 ml of water, decolorized with charcoal and applied onto Dowex 50 column (100 ml, H⁺-form). Non-aminic impurities were eluted with water (200 ml) and cyclic compounds were eluted by 5% aq. ammonia. Fractions containing amines were evaporated in vacuo and the residue was dissolved in 2 ml of water. The solution was applied onto Amberlite 50CG column (100 ml) and the column was eluted with water. The first two 100 ml fractions contained pure product, fractions 4 and 5 the pure inner lactam. Fractions containing pure compounds were evaporated and dissolved in 2 ml of conc. HCl. THF (50 ml) was slowly (3 h) dropped into the solutions with stirring. The solids were filtered, washed with THF and dried in vacuum over P₂O₅. Yield was 0.65-0.73 g of H₄do3a-P^{H}.2HCl.2H₂O (7.2HCl.2H₂O) and 0.15-0.20 g H₂do3a-lactam.2HCl.2H₂O (4.2HCl.2H₂O).

The compound was analysed using NMR.

³¹P NMR (1 M NaOD/D₂O)-22.4 ppm (¹J(PH)=500.0 Hz, ³J(PH)=9.2 Hz)

¹H NMR (1 M NaOD/D₂O): 2.77 ppm (dd, 2H, ²J(PH)=9.2 Hz, ³J(HH)=2.0 Hz, CH₂—P); 2.82-2.88 ppm (m, 16H, ring CH₂); 3.27 ppm (s, 2H, CH₂—COOH); 3.34 ppm (s, 4H, CH₂—COOH); 7.17 ppm (d, 1H, ¹J(PH)=500.0 Hz, P—H).

¹³C NMR (1 M NaOD/D₂O): 51.7-52.9 ppm (ring C); 57.5 ppm (¹J(PC)=99 Hz, C—P); 59.6 and 60.0 ppm (acetic CH₂); 178.9 and 179.3 ppm (COOH).

ESI/MS (positive): 425.1 (M+H⁺), 436.8 (M+Na⁺)

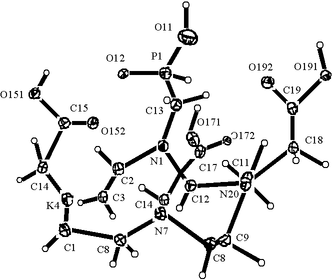

Molecular structure of DO3A-P^{H} in DO3A-P^{H}.HCl.5H₂O

### Example 7

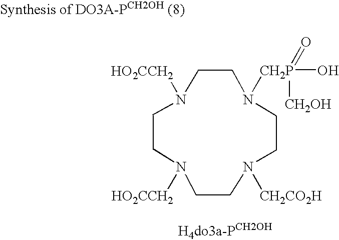

The same procedure as for synthesis of ligand 7 in Example 6 was used except that only 0.3 g (2 equiv.) of hypophosphorous acid and 10 equiv. (1.8 g) of aqueous formaldehyde was used. After purification, the solution was evaporated and the residue was dissolved in 1 ml of conc. HCl. THF was slowly dropped into the solution to give a white gum. It was several times triturated with THF to give a white powder that was dried in a vacuum desiccator. Yield of H₄do3a-P^{H}.2HCl.2H₂O (8.2HCl.2H₂O) was 0.47 g. The compound was analysed.

Elementary analysis (calc.): C 33.87 (34.11) H 7.20 (6.62) N 9.13 (9.95) Cl 13.10 (12.59)

³¹P NMR (D₂O, 90° C.): 33.2 ppm;

¹H NMR (KOD/D₂O, 90° C.): 2.45-2.76 ppm (bm, 16H, ring CH₂), 2.85 ppm (d, 2H, ²J(PH)=6.8 Hz), 3.03 pm (s, 2H, CH₂—COOH), 3.10 ppm (s, 4H, CH₂—COOH), 3.10 ppm (d, 2H, ²J(PH)=5.6 Hz)

ESI/MS (positive): 454.8 (M+H⁺), 476.9 (M+Na⁺)

### Example 8

Synthesis of DO3A-P^{CH2OH} (8)

0.60 g of DO3A-P^{H} (7) (1.1 mmol) was refluxed in 10 ml aq. HCl (1:1) and 5 ml of aq. CH₂O (37%). Yield after purification and drying as in Example 7 was 0.95 g.

### Example 9

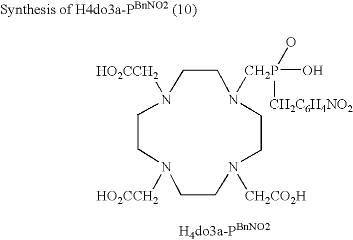

a) Synthesis of P-nitrobenzylphosphinic Acid (4-NO₂—C₆H₄)CH₂PO₂H₂ (PNBPA, 9) and Its Methyl and Ethylesters

Silyl ester P(OSiMe₃)(OEt)(CH(OEt)₂) (26.8 g, 0.1 mol) was dissolved in 100 ml of dry CH₂Cl₂. p-Nitrobenzylbromide (21.6 g, 0.1 mol) was dissolved in 100 ml of dry CH₂Cl₂ and slowly dropped into solution of silyl ester with stirring and cooling. It was left overnight at room temperature. MeOH (30 ml) was added and volatiles were removed using a rotavapor. The residue was dissolved in 25 ml of EtOH, 25 ml of conc. HCl was added and the solution was refluxed overnight. Solvents were evaporated in vacuo. The residue was dissolved in boiling water (100 ml) and 2 g of charcoal was added. After filtration and cooling in a refrigerator, the first crop of product crystallised and it was filtered off and dried on air. Further crops may be obtained after concentration of the filtrate. Overall yield wass 87%. The compound was analysed using NMR.

³¹P NMR (dmso-d₆): 31.1 ppm (¹J(PH)=541 Hz)

¹H NMR (CDCl₃): 3.48 ppm (d, 2H, ²J(PH)=7.2 Hz, CH₂); 7.42 ppm (d, 1H, ¹J(PH)=541 Hz); 7.57 (dd, 2H, ³J(HH)=8.8 Hz, ⁴J(PH)=2.0 Hz) and 8.23 ppm (d, 2H, ³J(HH)=8.8 Hz) for aryl

Methyl and ethyl esters were prepared by the same procedure as esters of MePO₂H₂ (2) (Example 2b) (following the procedure published by Y. R. Dumond et al., Supra). Purification was achieved by chromatography on SiO₂ instead of destillation.

b) Synthesis of P-nitrobenzylphosphinic Acid (4-NO₂—C₆H₄)CH₂PO₂H₂ (PNBPA, 9)

The compound was synthesised as compound 2 using 8.3 g (0.1 mol) NH₄H₂PO₂ and p-nitrobenzylbromide (13.4 g, 0.05 mol) and purified as described in Example 9a. Yield was 15%.

c) Reaction of DO3A with p-NO₂C₆H₄CH₂PO₂H₂ (9). Formation of DO3A-P^{BnNO2} (10)

0.5 g of H₃do3a (1.7 mmol) and 1 g p-NO₂C₆H₄CH₂PO₂H₂ (9) (5.1 mmol, 3 equiv.) was dissolved in 10 ml of azeotropic HCl in 50 ml flask equipped with a reflux condenser. The flask was flushed with argon. 0.2 ml of aqueous CH₂O (36%, 1 equiv.) was added into the flask. The reaction mixture was heated under gentle reflux for 24 h. Additionally, 0.5 ml of aqueous CH₂O (36%, 2.5 equiv.) was added and the mixture was refluxed for another 48 h. Solvents were removed using a rotary evaporator, the residue was dissolved in 2 ml of water, decolourised with charcoal and applied onto Dowex 50 column (100 ml, H⁺-form). Non-aminic impurities were eluted with water (200 ml) followed by water-EtOH mixture (1:1, 600 ml; removing of the starting acid and column by-products) and cyclic compounds were eluted by 5% aq. ammonia. Fractions containing amines were evaporated in vacuo and the residue was dissolved in 2 ml of water. The solution was applied onto Amberlite 50CG column (100 ml) and the column was eluted with water. The first two 100 ml fractions contained pure product, later fractions contained some inner lactam and unreacted H₃do3a. Fractions containing pure chelate were evaporated and the residue was dissolved in 1 ml water. THF (50 ml) was slowly (3 h) dropped into the solutions with stirring. The solid was filtered, washed with THF and dried in vacuo over P₂O₅. Alternatively, the water solution of the ligand was added dropwise to stirred absolute EtOH (100 ml), isolated and dried as above]. Yield was 0.38 g of DO3A-P^{BnNO2.}3H₂O. The compound was analysed.

Elemental analysis (calc): C41.96 (43.07), H 7.20 (6.57) N10.76 (11.41)

³¹P NMR (1 M NaOD): 34.0 ppm

¹H NMR (1 M NaOD/D₂O): 2.79-2.85 ppm (bm, 18H, N—CH₂—P and ring CH₂); 3.13 (d, 2H, ²J(PH)=15.6 Hz, P—CH₂-aryl); 3.25 ppm (b, 2H, CH₂—COOH); 3.30 ppm (s, 4H, CH₂—COOH); 6.76 (m, 2H, ⁴J(PH)=2 Hz, aryl) and 7.1 ppm, (m, 2H and 2H, aryl).

¹³C NMR (1 M NaOD/D₂O): 41.48 ppm (d, ¹J(PH)=75.6 Hz, P—C-aryl); 51.67, 52.17, 52.59 and 52.60 ppm (ring C); 55.35 ppm (d, ¹J(PC)=96.8 Hz, N—C—P); 59.57 and 59.90 ppm (two s, acetic CH₂); 178.67 and 1179.25 ppm (COOH); 125.06 ppm; 132.15 ppm, d, ³J(PC)=4.6 Hz; 145.39 ppm, d, ²J(PC)=−7.6 Hz; 147.30 ppm, d, ⁵J(PC)=3.0 Hz

ESI/MS (positive): 560.1 (M+H⁺)

### Example 10

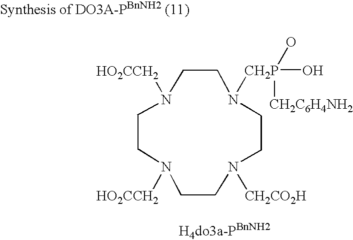

The nitro compound 10 (0.1 g) was dissolved in 5 ml of water, the solution was acidified with 0.5 ml of formic acid and 0.01 g of 10% Pd/C was added. The mixture was kept under hydrogen (atmospheric pressure) with stirring for 48 h. Catalyst was filtered off. Solvents were removed using a rotary evaporator, the residue was dissolved in 2 ml of water, decolourised with charcoal and applied onto Dowex 50 column (100 ml, H⁺-form). Nbn-aminic impurities were eluted with water (200 ml) followed by water (500 ml) and cyclic compounds were eluted by 5% aq. ammonia. Fractions containing amines were evaporated in vacuo and the residue was dissolved in 2 ml of water. The solution was applied onto Amberlite 50CG column. (100 ml) and the column was eluted with water. The first two 100 ml fractions containing pure product were evaporated and the residues were combined and dissolved in 1 ml of water. THF (50 ml) was slowly (3 h) dropped into the solution while stirring. The solid was filtered, washed with THF and dried in vacuo over P₂O₅. Alternatively, the water solution of the ligand was added dropwise to stirred absolute EtOH (100 ml), isolated and dried as above. Yield was 0.087 g of DO3A-P^{BnNH2}.3H₂O. The compound was analysed.

Elementary analysis (calc.): C45.70 (45.28) H 7.19 (7.25) N 11.67 (12.00)

ESI/MS (positive): 530.2 (M+H⁺)

³¹P NMR (1 M NaOD, 75° C.): 37.5 ppm

¹H NMR (1 M NaOD/D₂O, 25° C.): 2.79 ppm (b, 4H, CH₂—P—CH₂); 2.57-2.72 ppm (m, 16H, ring CH₂); 2.57-3.12 ppm (b, 2H, CH₂—COOH); 3.34 ppm (s, 4H, CH₂—COOH); 6.76 and 7.1 ppm, (m, 2H and 2H, aryl).

¹³C NMR (1 M NaOD/D₂O, 75° C.): 52.88, 53.21 and 53.53 ppm (ring C); 41.26 ppm (d, ¹J(PC)=81.3 Hz, Ar—C—P); 55.4 ppm (d, ¹J(PC)=92.3 Hz, N—C—P); 60.97 ppm (b, acetic CH₂); 180.66 and 180.97 ppm (COOH); 118.89 ppm, d, ⁴J(PC)=2.31 Hz; 128.20 ppm, d, ²J(PC)=7.2 Hz; 132.92 ppm, d, ³J(PC)=5.3 Hz; 146.63 ppm, d, ⁵J(PC)=2.6 Hz

### Example 11

Synthesis of DO3A-P^{BnNH2} (11)

EtOH:conc. aq. NH₃ (1:1) mixture was saturated with H₂S and the nitro compound 10 was added (0.1 g). The mixture was refluxed for 6 h. During that time, the solution was saturated 4 times with H₂S. Solvents were evaporated from the suspension. The residue was dissolved 5 times in AcOH and evaporated (removing of H₂S and coagulation of sulphur), dissolved in water and solution was filtrated through a plug of charcoal. Purification on Amberlite 50CG (elution with water) gave product (about 0.031 g after evaporation and crystallisation as in Example 10, eluted as the second band) and a large amount of the starting acid.

### Example 12

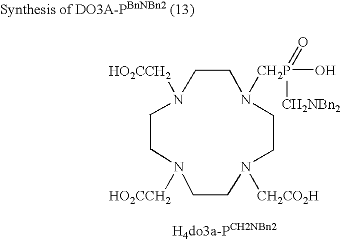

a) Synthesis of (PhCH₂)₂NCH₂PO₂H₂ (12) and Its Esters

3.95 g (0.02 mol) of (PhCH₂)₂NH and 2.64 g of 50% aqueous H₃PO₂ (0.03 mol) was dissolved in 25 ml of water. Aqueous formaldehyde (30%, 1.2 g, 0.04 mol) was slowly dropped into the solution at a temperature of 100° C. It was refluxed for 5 h. After cooling, volatiles were removed in vacuo. The residue was dissolved in minimum amount of water and purified on Dowex 50. Acids were removed by water elution and the product was eluted with 1% aqueous ammonia. Fractions containing the product were evaporated and trituration of residual oil with dry THF gave 35% of white solid. The compound was analysed using NMR.

³¹P NMR (D₂O): 24.3 ppm (¹J(PH) 514 Hz)

¹H NMR (D₂O): 2.68 ppm (dd, 2H, ²J(PH)=10.4 Hz, ³J(HH)=2.0 Hz, CH₂P); 3.83 (s, 2H, CH₂Ph); 6.99 (dt, 1H, ¹J(PH)=514 Hz, ³J(HH)=2 Hz); 7.38-7.44 (m, 5H, aryl)

Methyl and ethyl esters on phosphorus atom were prepared by the same procedure as esters of MePO₂H₂ (2) (Example 2b) and purified by chromatography on SiO2 instead of destillation (following the procedure published by Y. R. Dumond et al., Supra).

b) Reaction of DO3A with Bn₂NCH₂PO₂H₂ (12). Formation of DO3A-P^{CH2NBn2} (13)

0.65 g DO3A (1.5 mmol) and 1.86 g Bn₂NCH₂PO₂H₂ (12) (6.7 mmol, 4.5 equiv.) were dissolved in 10 ml of azeotropic HCl in 50 ml flask equipped with the reflux condenser. The flask was flushed with argon. 0.5 ml of aqueous CH₂O (36%, 3 equiv.) was added into the flask. The reaction mixture was heated under gentle reflux for 24 h. Additional 0.5 ml of aqueous CH₂O (36%, 3 equiv.) was added and the mixture was refluxed for another 6 h. Solvents were removed using a rotary evaporator, the residue was dissolved in 2 ml of water, decolourised with charcoal and applied onto Dowex 50 column (100 ml, H⁺-form). Non-aminic impurities were eluted with water (200 ml) and cyclic compounds were eluted by 5% aq. ammonia. Fractions containing amines were evaporated in vacuo and the residue was dissolved in 2 ml of water. The solution was applied onto Amberlite 50CG column (100 ml) and the column was eluted with water. The first four 100 ml fractions contained pure product. The fractions containing pure chelate were evaporated and the residue was dissolved in 1 ml of water. THF (100 ml) was slowly (5 h) dropped into the solutions while stirring. The solid was filtered, washed with THF and dried in vacuo over P₂O₅. Yield was 0.95 g of D0₃A-P^{CH2NBn}.

The compound was analysed using NMR.

### Example 13

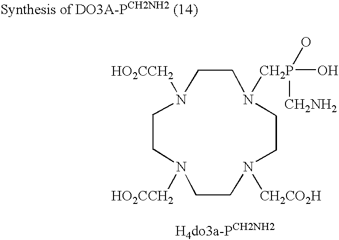

The dibenzylamino ligand 13 (0.15 g) was dissolved in 10 ml of water, the solution was acidified with 0.5 ml of formic acid and 0.02 g of 10% Pd/C was added. The mixture was kept under hydrogen (atmospheric pressure) and stirred for 24 h. Catalyst was filtered off. Solvents were removed using a rotary evaporator, the residue was dissolved in 2 ml of water, decolourised with charcoal and applied onto Dowex 50 column (100 ml, H⁺-form). Non-aminic impurities were eluted with water (200 ml) followed by water (500 ml) and cyclic compounds were eluted by 5% aq. ammonia. Fractions containing amines were evaporated in vacuo and the residue was dissolved in 2 ml of water. The solution was applied onto Amberlite 50CG column (100 ml) and the column was eluted with water. The first three 100 ml fractions containing pure product were evaporated and the residue was dissolved in 2 ml of conc. HCl. THF (100 ml) was slowly (5 h) dropped into the solutions while stirring. The solid was filtered, washed with THF and dried in vacuo over P₂O₅. Yield was 0.073 g of DO3A-P^{CH2}NH2.2HCl.?H₂O (13.2HCl.?H₂O). The compound was analysed using NMR.

### Example 14

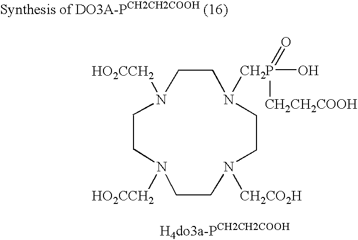

a) Synthesis of HOOCCH₂CH₂PO₂H₂ (15) and Its Esters

Ethyl acrylate (2.00 g, 0.02 mol) and ester (3.92 g, 0.02 mol) were dissolved in 20 ml of toluene and NaOEt solution (made from 0.46 g Na in 10 ml EtOH and 10 ml toluene) was added dropwise. The mixture was stirred for 20 h at room temperature. Solvent was removed using a rotavapor and protected ester was hydrolysed in refluxing aqueous HCl. After evaporation in vacuo, the product was purified on Dowex 50 column in H⁺ cycle. The acid was eluted with water and, after evaporation in vacuo, the product was obtained as a clear oil in 75% yield. The compound was analysed using NMR.

³¹P NMR (CDCl₃): 33.5 ppm;

¹H NMR (CDCl₃): 2.03-2.12 ppm (m, 2H), 2.60-2.68 ppm (m, 2H), 7.22 (dt, 1H, ¹J(PH)=562 Hz, ³J(HH)=2.0 Hz

Methyl and ethyl esters on phosphorus atom were prepared by the same procedure as esters of MePO₂H₂ (2) (Example 2b) and purified by chromatography on SiO₂ instead of destillation (following the procedure published by Y. R. Dumond et al., Supra.

b) Synthesis of HOOCCH₂CH₂PO₂H₂ (15) (Following the Procedure Published by A. E. Wroblewski et al. J. Am. Chem. Soc. 1996, 118, 10168)

Methyl acrylate (2.15 g, 0.025 mol) was dissolved in 20 ml of HC(OMe)₃ and the mixture was kept at room temperature for 24 h. Volatiles were removed using a rotavapor and residual oil was heated at 40° C. at vacuum (0.2 torr) for 15 h. The residue consists of almost pure MeOOCCH₂CH₂P(O)(H)(OMe). It was dissolved in azeotropic HCl and refluxed overnight. The acid was purified as in Example 14a.

c) Reaction of DO3A with HOOCCH₂CH₂PO₂H₂ (15). Formation of DO3A-P^{CH2CH2COOH} (16)

0.65 g DO3A (1.5 mmol) and 0.93 g HOOCCH₂CH₂PO₂H₂ (15) (6.7 mmol, 4.5 equiv.) were dissolved in 10 ml of azeotropic HCl in 50 ml flask equipped with the reflux condenser. The flask was flushed with argon. 0.5 ml of aqueous CH₂O (36%, 3 equiv.) was added into the flask. The reaction mixture was heated under gentle reflux for 24 h. Additionally, 0.5 ml of aqueous CH₂O (36%, 3 equiv.) was added and mixture was refluxed for another 6 h. Solvents were removed using a rotary evaporator, the residue was dissolved in 2 ml of water, decolourised with charcoal and applied onto Dowex 50 column (100 ml, H⁺-form). Non-aminic impurities were eluted with water (200 ml) and cyclic compounds were eluted by 5% aq. ammonia. Fractions containing amines were evaporated in vacuo and the residue was dissolved in 2 ml of water. The solution was applied onto Amberlite 50CG column (100 ml) and the column was eluted with water. The first four 100 ml fractions contained pure product. The fractions containing pure chelate were evaporated and the residue was dissolved in 1 ml of water. THF (100 ml) was slowly (5 h) dropped into the solutions while stirring. The solid was filtered, washed with THF and dried in vacuo over P₂O₅. Alternatively, the water solution of the ligand was added drop-wise to stirred EtOH (100 ml), isolated and dried as above. Yield was 0.67 g of DO3A-P^{CH2CH2COOH.}3H₂O. The compound was analysed.

Elementary analysis (calc.): C 39.15 (39.27) H 7.32 (7.14) N 10.10 (10.18)

³¹P NMR (D₂O, 90° C.): 37.1 ppm;

¹H NMR (D₂O, 90° C.): 2.43-2.50 ppm (m, 2H), 3.10-3.17 ppm (m, 2H), 3.78-4.03 ppm (m, 16H, ring CH₂), 4.24 ppm (s, 2H, CH₂—COOH), 4.27 ppm (s, 4H, CH₂—COOH);

¹³C NMR (D₂O, 90° C.): 26.65 ppm (d, CH₂CH₂P, ¹J(PC)=94.2 Hz); 27.59 ppm (CH₂COOH), 50.19-51.41 ppm (azacycle carbons), 52.51 (d, NCH₂P, ¹J(PC)=85.4 Hz), 55.62 ppm and 57.72 ppm (acetate carbons), 171.72 and 172.53 (pendant carboxyl), 177.45 (d, side-chain carboxyl group, ³J(PC)=13.8 Hz)

ESI/MS (positive): 497.1 (M+H⁺); (negative) 495.3 (M−H⁺)]

### Example 15

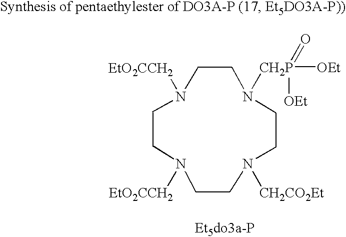

0.5 g (1.16 mmol) of triethylester of DO3A and 0.48 g (3.48 mmol) diethylphosphite were dissolved in 15 ml of dry benzene and paraformaldehyde (0.14 g, 4 equiv.) was added to refluxing solution in small portions over 2 h. Water was removed using a Dean-Stark apparatus. Mixture was refluxed overnight. Solvents were removed using a rotavapor and the residue was dissolved in EtOH. The solution was decolourized by charcoal and purified by chromatography on SiO₂ column (EtOH:25% aq. NH3=15:1). Fractions containing the pure product were evaporated resulting in a slightly yellow oil (57%). The compound was analysed using NMR.

### Example 16

Synthesis of Pentaethylester of DO3A-P (17, Et₅DO3-P))

0.5 g (1.16 mmol) of triethylester of DO3A and 0.66 g (4 mmol) triethylphosphite were dissolved in 15 ml of dry benzene and paraformaldehyde (0.14 g, 4 equiv.) was added to refluxing solution in small portions over 2 h. Mixture was refluxed overnight. Solvents were removed using a rotavapor and the residue was dissolved in EtOH. The solution was decolourized by charcoal and purified by chromatography on SiO₂ column (EtOH:25% aq. NH₃=15:1). Fractions containing the pure product were evaporated resulting in a slightly yellow oil (84%).

### Example 17

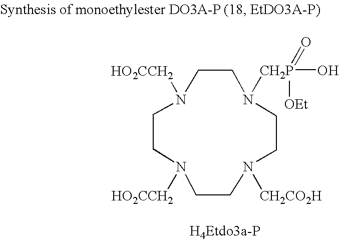

0.65 g of ester 17 was dissolved in 10 ml of 1 M aqueous NaOH and refluxed overnight. Water was evaporated and residue was dissolved in 3 ml of water. The solution was applied on Dowex 1 (OH⁻-form) column and eluted with water to remove sodium ions. Product was obtained by elution with 5% aqueous AcOH. Fractions containing product were evaporated to dryness and dissolved in water and evaporated several times to remove excess of AcOH. The residue was dissolved in 1 ml of water and product 18 precipitated by adding anhydrous EtOH (76%). The compound was analysed using NMR.

### Example 18

Synthesis of DO3A-P (1)

Compound 17 (0.65 g) was dissolved in azeotropic HCl and refluxed overnight. The reaction mixture was purified as in Example 1 to give trihydrate of 1 in 92% yield. The batch of compound 1 resulted in identical spectroscopic data as the batch in Example 1.

### Example 19

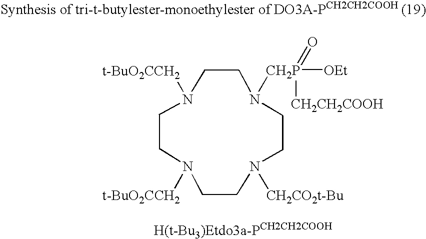

1.00 g (1.94 mmol) of tri-t-butylester of DO3A and 1.33 g (8 mmol) of P-ethylester of acid 15 were dissolved in 20 ml of dry benzene and paraformaldehyde (0.14 g, 4 equiv.) was added to refluxing solution in small portions over 3 h. Water was removed using a Dean-Stark apparatus. Mixture was refluxed overnight. Solvents were removed using a rotavapor and the residue was dissolved in EtOH. The solution was decolourized by charcoal and purified by chromatography on SiO₂ column (EtOH:25% aq. NH3=10:1). Fractions containing the pure product were evaporated resulting in a slightly yellow oil (73%). The compound was pure enough for coupling to targeting moieties. The compound was analysed using NMR.

### Example 20

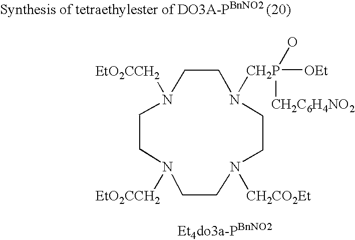

0.5 g (1.16 mmol) triethylester of DO3A and 1.15 g (5 mmol) of ethylester of acid 8 were dissolved in 15 ml of dry benzene and paraformaldehyde (0.21 g, 6 equiv.) was added to refluxing solution in small portions over 5 h. Water was removed using a Dean-Stark apparatus. Mixture was refluxed overnight. Solvents were removed using a rotavapor and the residue was dissolved in EtOH. The solution was decolourized by charcoal and purified by chromatography on SiO₂ column (EtOH:25% aq. NH3=10:1). Fractions containing the pure product were evaporated resulting in a yellow oil (45%). The compound was analysed using NMR.

### Example 21

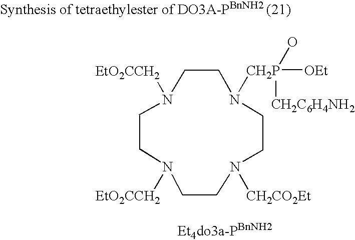

The nitro chelate 20 (0.2 g) was dissolved in 5 ml of EtOH, the solution was acidified with 0.5 ml of formic acid and 0.02 g of 10% Pd/C was added. The mixture was kept under hydrogen (atmospheric pressure) and stirred for 24 h. Catalyst was filtered off. Solvents were removed using a rotary evaporator and the residue was purified by chromatography on SiO₂ column (EtOH:25% aq. NH₃=10:1). Fractions containing the pure product were evaporated resulting in a yellow oil (82%). The compound was analysed using NMR.

### Example 22

Synthesis of Tetraethylester of DO3A-P^{R} (22, R=—CH₂C₆H₄-4-(NHC(O)CH₂Br))

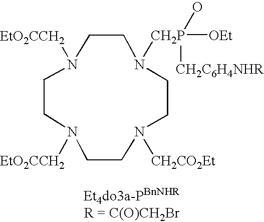

The amino chelate 21 (0.15 g) was dissolved in 30 ml of dry acetonitrile and 1.5 g of finely powdered dry K₂CO₃ was added. Bromoacetylbromide (1.1 equiv.) was slowly dropped into vigorously stirred suspension. The mixture was stirred a room temperature for 20 h. It was filtered and evaporated to dryness. After chromatography on SiO₂ product 22 was obtained in 65% yield. The compound was analysed using NMR.

Synthesis of Other Precursors

### Example 23

Synthesis of HP(O)(OEt)(CH(OEt)₂) (23)

20.1 g (0.3 mol) of anhydrous phosphinic acid was dissolved in 150 ml of HC(OEt)₃ and 4 ml of water. After dissolving of all solids, 4.5 ml of F₃CCOOH was dropped during 5 min under stirring and cooling using a cold water bath. The mixture was left at room temperature for a week. Volatiles were removed using a rotavapor (bath temperature max. 40° C.) and the remaining liquid was dissolved in 180 ml of CH₂Cl₂. The solution was extracted with aqueous phosphate buffer (21 g of Na₂HPO₄<12H₂O in 180 ml of water). Organic phase was dried with anhydrous Na₂SO₄ and filtered. Solvent was removed using a rotavapor (bath temperature max. 40° C.) and any residual solvents were distilled off at lower pressure (1 torr) at temperature around 40° C. The target compound was distilled with a short column at 65-73° C./0.25 torr. Yield was 68% (>98% purity as determined by ³¹P NMR spectroscopy, δ_{P}=27.8 ppm (neat).

### Example 24

Synthesis of P(OSiMe₃) (OEt) (CH(OEt)₂) (24)

Compound 23 (28.6 g, 0.146 mol) was dissolved in 38 ml of hexamethyldisilazane and refluxed under low flow of argon for 6 h. Reaction mixture was cooled to room temperature and carefully fractionated under pressure 1 torr with a short column. Fraction boiling at 52-55° C./1 torr was collected to give 91% yield of the desired ester as an air and moisture sensitive liquid.

³¹P NMR (dry CDCl₃): 146.8 ppm; ²⁹Si NMR (neat): 17.3 ppm

¹H NMR (dry CDCl₃): 1.25-1.30 ppm (m, 6H, CH(OCH₂CH₃)₂); 1.39 ppm (t, 3H, ³J(HH)=7.2 Hz, POCH₂—CH₃), 3.68-3.77 ppm (m, 2H); 3.82-3.90 ppm (m, 2H); 4.09-4.27 ppm (m, 2H); 4.72 ppm (dd, 1H, ²J(PH)=7.6 Hz, ³J(HH)=1.6 Hz, P—CH); 6.95 ppm (dd, 1H, ¹J(PH)=554 Hz, ³J(HH)=1.6 Hz, P—H)

### Example 25

Synthesis of HP(O)(OMe)(CH(OMe)₂) (25)

Prepared as described for compound (23) from 20.1 g (0.3 mol) of anhydrous hypophosphorus acid, 130 ml HC(OMe)₃, 4 ml of water and 4.5 ml CF₃COOH. Yield 65% (>95% purity, b.p. 68-73° C./0.25 torr). The compound was analysed using NMR (δ_{P}=29.8 ppm (neat)).

### Example 26

Synthesis of P(OSiMe₃)(OMe)(CH(OMe)₂) (26)

Synthesised as described for compound (24) from 25 g (0.18 mol) of 25 in a yield of 92% (b.p. 38-41° C./1 torr). The compound was analysed using NMR.

### Example 27

Synthesis of HP(O)(OiPr)(CH(OiPr)₂) (27)

Prepared as described for compound (23) from 20.1 g (0.30 mol) of anhydrous hypophosphorus acid, 170 ml HC(OiPr)₃, 4 ml of water and 4.5 ml CF₃COOH. Yield was 78% (>95% purity, b.p. 102-108° C./0.25 torr). The compound was analysed using NMR.

### Example 28

Synthesis of P(OSiMe₃)(OiPr)(CH(OiPr)₂) (28)

Synthesised as described for compound (24) from 28 g (0.118 mol) of 27 in a yield of 85% (b.p. 62-5° C./1 torr). The compound was analysed using NMR.

### Example 29

Synthesis of P-nitrophenylphosphinic Acid 4-NO₂—C₆H₄—PO₂H₂ (29) (Following the Procedure Published by J.-L. Montchamp J. Am: Chem. Soc. 2001, 123, 510)

A mixture of anilinium salt of H₃PO₂ (0.26 g, 3.5 mmol) and 4-NO₂—C₆H₄-l (0.75 g, 3 mmol) was dissolved in 10 ml of DMF and 50 mg of Pd(PPh₃)₄ (catalyst) and 1 ml of Et₃N was added. The reaction mixture was stirred at 90° C. for 5 h. DMF was removed in vacuo and water was added to the residue, acidified to approximately pH 1, saturated with NaCl and extracted 3 times with ethylacetate. The organic fraction was collected, dried (MgSO₄) and evaporated to give 74% of product.

Methyl and ethyl esters on phosphorus atom were prepared by the same procedure as esters of MePO₂H₂ (2) (Example 2b) and purified by chromatography on SiO₂ instead of destillation (following the procedure published by Y. R. Dumond et al., Supra).

### Example 30

Synthesis of HOOCCH₂PO₂H₂ (30)

Silyl ester P(OSiMe₃)(OEt)(CH(OEt)₂) (24) (26.8 g, 0.1 mol) (13.4 g, 0.05 mol) was dissolved in 100 ml of dry CH₂Cl₂. Ethyl bromoacetate (8.35 g, 0.05 mol) was dissolved in 50 ml of dry CH₂Cl₂ and slowly dropped into solution of silyl ester while stirring and cooling. It was left overnight at room temperature. MeOH (30 ml) was added, solution was stirred for 30 min at room temperature. It was filtered and volatiles were removed using a rotavapor. The residue was dissolved in 25 ml of EtOH, 25 ml of conc. HCl was added and the solution was refluxed overnight. Solvents were removed in vacuo and HCl from the residue was removed by repeated evaporation with water. Residual oil was pure enough for next reactions or for synthesis of esters. The compound was analysed using NMR.

Methyl and ethyl esters on phosphorus atom were prepared by the same procedure as esters of MePO₂H₂ (2) (Example 2b) and purified by chromatography on SiO₂ instead of destination (following the procedure published by Y. R. Dumond et al., Supra).

### Example 31

Synthesis of NH₂CH₂CH₂CH₂PO₂H₂ (31)

Acrylonitrile (1.06 g, 0.02 mol) and ester HP(O)(OEt)(CH(OEt)₂ (23) (3.92 g, 0.02 mol) were dissolved in 20 ml toluene and NaOEt solution (made from 0.46 g Na in 10 ml EtOH and 10 ml toluene) was added dropwise. The mixture was stirred for 20 h at room temperature. Solvents were removed using a rotavapor and residue was dissolved in 50 ml of dry EtOH. 1.5 g (0.04 mol) of NaBH₄ was added in small portions to stirred solution of nitrile. It was stirred overnight. Excess of borohydride was destroyed by 10 ml of water and 50 ml of conc. HCl. Mixture was refluxed overnight. It was cooled and volatiles were removed in vacuo. The residue was dissolved in small amount of water and applied on column with Dowex 50 (H⁺-cycle). After elution with water, the product was obtained when eluted with 0.5% aqueous ammonia. Fractions containing the product were evaporated in vacuo and remaining oil was triturated with dry THF to get 71% of the solid product.

The compound was analysed using NMR.

### Example 32

Synthesis of PhCH₂NHCH₂CH₂PO₂H₂ (32)

4.90 g (0.025 mol) of ester HP(O)(OEt)(CH(OEt)₂ (23) and 3.33 g (0.025 mol) of N-benzyl-aziridine was dissolved in 50 ml of dry toluene. Solution of NaOEt (made from 0.06 g Na in 5 ml of dry EtOH) was dropped in the solution and mixture was refluxed for 48 h. Solvent was removed in vacuo and the residue was dissolved in 50 ml of dry EtOH. 50 ml of conc. aqueous HCl was added and solution was refluxed overnight. Purification on Dowex 50 column was done as described for compound 12 in Example 12 produced 78% of white solid.

### Example 33

Synthesis of (PhCH₂)₂NCH₂CH₂PO₂H₂ (33) and Its Esters

Acid 32 (1.00 g, 5 mmol) was dissolved in 20 ml of water and pH was increased by addition of aqueous NaOH. Benzoylchloride (1.00 g, 8 mmol) was dropped into the solution while stirring. After 2 h, the mixture was acidified to approximately pH 2 using aqueous HCl. Precipitated solid was filtered, washed with water and dried in vacuo. The solid was dissolved in dry THF and 10 ml 1 M BH₃.SMe₂ (0.01 mol) was added in small portions. The solution was stirred for 1 h at room temperature and than refluxed for 5 h. Solvent was removed in vacuo and the residue was dissolved in azeotropic HCl and refluxed for 5 h. Volatiles were removed using a rotavapor and residual oil was purified on DOWEX 50 column as compound 12 in Example 12 to result in pure compound 33 in a yield of 53%.

The compound was analysed using NMR.

Methyl and ethyl esters on the phosphorus atom were prepared by the same procedure as esters of MePO₂H₂ (2) (Example 2b) and purified by chromatography on SiO₂ instead of destillation (following the procedure published by Y. R. Dumond et al., Supra).

Complexation of Metal Ions

### Example 34

Synthesis of Gadolinium(III) Complex of DO3A-P (34)

Gd₂O₃ (0.037 g, 0.01 mmol) was dissolved in 2 ml of conc. HCl and the solution was evaporated to dryness in vacuo. The residue was dissolved in Water (2 ml) and 0.10 g (0.20 mmol) of hydrate of DO3A-P (1) was added. The solution was stirred at 40° C. for 30 min and pH was slowly increased by addition of diluted aqueous NaOH solution to about 8. Any precipitated gadolinium hydroxide was centrifuged and supernatant was purified on Amberlite 50 (H⁺-form) column by elution with water. Fractions containing complex were evaporated to dryness in vacuo. The residue was dissolved in 1 ml of water and the solution was slowly dropped into 30 ml of anhydrous EtOH to give 110 mg of slightly hygroscopic solid.

### Example 35

Synthesis of Gadolinium(III) Complex DO3A-P^{Bn} (35)

The same procedure as for compound 34 in Example 34 was used except that 0.16 g (0.195 mmol) of acid 5 adduct was used to give 118 mg of the complex after purification.

### Example 36

Synthesis of Yttrium(III) Complex of DO3A^{BnNH2} (36)

The same procedure as for compound 34 in Example 34 was used except that 0.21 g (0.195 mmol) of acid 11 adduct and Y203 (0.5 equiv.) was used to give 135 mg of the complex after purification.

### Example 37

Synthesis of Isothiocyanatobenzylphosphinic Acid Derivative of DO3A.

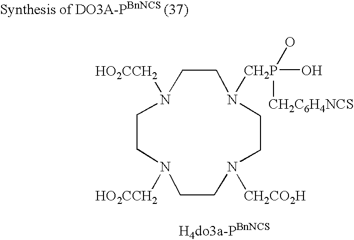

DO3A-P^{BnNH2} (11) (200 mg, 0.38 mmol) was dissolved in 3 ml of water. Solution was acidified with hydrochloric acid to pH 2-3, afterwards solution of thiofosgen (37 μl 90% (by GC) CSCl₂ in 2 ml CCl4) was added and reaction mixture was shaken for 12 h in the dark at room temperature. Water phase was separated and washed twice with 2 ml of CCl₄ and twice with 1 ml of Et₂O and consequently, evaporated in vacuum (max. 30° C.) to glass. The glass-crude product (95% according to NMR results) was ground and characterised by ¹H, ³¹P NMR, IR and UV spectroscopies. This compound is suited for coupling to the ε-amino group of lysines.

Isolated as approx. DO3A-P^{BnNCS}.4HCl.

Elementary analysis (calc.): C 38.01 (38.51) H 5.93 (5.34) N 10.10 (9.76) S 4.54 (4.47)

³¹P NMR (D₂O): 29.8 ppm;

¹H NMR (D₂O): 2.87-3.90 ppm (broad m, 24H, ring CH₂ and pendant CH₂), 7.20+7.23 (two m, 2H+2H, aromatic ring);

¹³C NMR (D₂O): 53.6 (d, ¹J(PC)=79.8 Hz, P—CH₂-benzyl), 54.9-56.7 ppm (azacycle carbons), 57.5 (d, NCH₂P, ¹J(PC)=82.0 Hz), 58.3 ppm and 59.7 ppm (acetate carbons), 134.5+136.2+139.3 ppm (d+d+s, J(PC)=3.8+5.3 Hz, aromatic ring), 136.8 (bs, NCS), 173.6 and 179.7 (pendant carboxyl)

ESI/MS (positive): 572.4 (M+H⁺); (negative) 495.3 (M−H⁺)

IR: 2100 cm⁻¹ (b, uₐₛ-NCS)

UV: 273 and 284 nm (aromatic ring and —NCS)

For covalent attachment of DO3A-P to SH-groups of cysteins, (S)—N-4-[2,3-Bis {bis(carboxyxmethyl)amino}-propyl]phenyl bromoacetamid derivatives of DO3A-P can be synthesized using procedures known in the art.

Formation of Bioconjugates and Complexation

### Example 38

Preparation of Solutions and Vessels:

Before processing, all vessels, reaction solutions and buffers have to be prepared as “low metal containing solutions” to avoid blockade of the metal binding portion of the chelate with inappropriate metal ions. Therefore before use, all reaction solutions and buffers are chromatographed through a chelating sepharose (Pharmacia) to remove trace amounts of contaminating metals. The low metal reaction solutions have to be stored in sterile polypropylene or polyethylene vessels until use.

### Example 39

Conjugation Reaction of D0₃A-P^{BnNCS} with glycine.

Crude DO3A-P^{BnNCS} (37) (50 mg, 69.6 μmol) was dissolved in water (0.5 ml) thereafter a solution of glycine was added (98 μl of 0.8 M solution in water) and pH was adjusted to 8 by addition of diluted potassium hydroxide solution. Reaction mixture was stirred for 6 h in the dark and was evaporated to glass and powdered. Crude product (90%) was characterised by ¹H, ³¹P NMR and IR spectroscopies.

### Example 40

Formation of ⁹⁰Y-yttrium and ⁸⁸Y-yttrium-DO3A-P Complexes

Purified DO3A-P^{BnNH2} was dissolved in demineralised water at a concentration of 9×10⁻⁵ mol/l. 0.1 ml of this solution were transferred into a small reaction vial (PE). 0.1 ml ⁹⁰Y-yttrium chloride (YCl₃) in 0.1 M HCl and 0.1 ml ammonium acetate buffer, pH 5.7, were added. The reaction solutions were mixed well. pH values were measured continuously while preparing the solution. 24 identical solutions were prepared accordingly and stored at 25° C. and 37° C. respectively.

Samples were taken after 15 min, 30 min, 45 min, 60 min, 90 min and 120 min and analyzed by thin layer chromatography using silica gel (POLYGRAM SIL G/UV₂₅₄) or, preferably, paper (Whatman No. 1) as solid phase. TLC was run using either solvent I: 0.1 N ammonium acetate solution or solvent II: 3% sodium chloride solution as developing solution. In parallel, samples (20 μl) were analyzed by gel filtration using HPLC. The HPLC-system comprised a gamma detector (Berthold LB 506) and a UV/VIS spectrometer (Waters 486) installed in two flow through cells, respectively. Both methods showed a fast complex formation of a ⁹⁰Y-yttrium-DO3A-complex comprising two phases.

The first phase of complex formation starts immediately as a reaction of yttrium (or other trivalent metal ions) with the protonated groups of the DO3A-P molecule under acidic conditions (pH 3-4). During the second phase, which is slower than the first phase and takes place at higher pH-values (pH 5-6), metallic ions (trivalent metal ions and lanthanides) are transferred into the inner part of the DO3A-P molecule while protons are eliminated from the nitrogen atom. The second step is catalyzed by OH-groups.

Temperature- and time-dependent studies have shown a very short reaction time for the complex formation of a ⁹⁰Y-Yttrium-DO3A-complex. Surprisingly, even at 25° C. immediately after addition of ⁹⁰Y-Yttrium 88% binding of ⁹⁰Y to DO3A-P^{BnNH2} was achieved reaching an optimal binding of 97% within 15 min (see Table 4).

Furthermore, the effect of pH and ligand concentration on radiochemical yield was evaluated. Table 5 summarizes the results relating to the variation of pH between pH 2.0 and pH 8.9 while maintaining a constant ratio of DO3A-P^{BnNH2} and Y of 3:1 and a reaction time of 60 min at 25° C. Accordingly, best labeling results are achieved at pH values of 4.9-8.0. Table 6 summarizes the results relating to the variation of the ligand concentration DO3A-P^{BnNH2}: Y between 1:1 up to 7:1 while maintaining a constant pH range (pH 5.2) and reaction time of 60 min at 25° C.

Under these conditions, an optimal labeling of the complex (94%) is already achieved at a rate of DO3A-P^{BnNH2}:Y=1:1.

Similar reaction kinetics are observed with other lanthanides such as ⁸⁸Y-Yttrium suited for therapeutic purposes. Corresponding data are shown in Tables 7-8.

### Example 41

Animal Studies to Evaluate Biodistribution and Elimination of ⁸⁸Y-DO3A-P Complex

The chelate DO₃A-P^{BnNH2} was radio lablelled using carrier free ⁸⁸Y-yttrium (in form of yttriumchloride (YCl₃), see example 40 above) resulting in a respective ⁸⁸Y-DO3A-P^{BnNH2}-complex. Radiochemical purity of this complex was tested using thin layer chromatography. Its pharmacokinetic characteristics were evaluated in animal studies.

Biodistribution and Elimination Studies in Animals

Biodistribution and elimination studies of ⁸⁸Y-DO3A-P^{BnNH2} complex were performed in Wistar SPF rats. The following in vivo and in vitro assays were carried out:

1. Determination of biodistribution of 88Y-DO3A-P^{BnNH2} complex in organs

2. Determination of elimination (mode and rate) of ⁸⁸Y-DO3A-P^{BnNH2} complex from the organism

3. Determination of the in vitro binding capacity of ⁸⁸Y-DO3A-P^{BnNH2} complex to human plasma proteins

4. Determination of the stability of 88Y-DO3A-P^{BnNH2} complex in human plasma

Results

1. The organ distribution of the ⁸⁸Y-DO3A-P^{BnNH2} complex, based on the measured ⁸⁸Y-yttrium activity in single organs, systems and tissues of the animals as well as activity concentration within single organs, systems and tissues measured 5 min, 60 min, 120 min and 24 h after intravenous application of the ⁸⁸Y-DO3A-P^{BnNH2} complex into the vena saphena are summarized in Tables 1, 2 and 3. (Single values are mean values of 4 animals each).

2. Mode and rate of elimination of ⁸⁸Y-DO3A-P^{BnNH2} complex from the organism as determined by cumulative excretion of radioactivity in intervals of 0-2 h and 0-24 h respectively after intravenous injection of ⁸⁸Y-DO3A-P^{BnNH2} complex into the vena saphena of Wistar SPF rats are summarized in Table 4.

3. Binding of the 88Y-DO3A-P^{BnNH2} complex to human plasma proteins was evaluated at 37° C. using equilibration dialysis or ultrafiltration. 10.2±2.3% or 3.7±3.2% were bound to plasma proteins, respectively. Pharmacokinetically, reversible binding is of no importance.

4. Stability of the ⁸⁸Y-DO3A-P^{BnNH2} complex was determined in human plasma at 37° C. over 14 days using standardised in vitro conditions. The ⁸⁸Y-DO3A-P^{BnNH2} complex was found to be highly stable. Dissociation of the radionuclide ⁸⁸Y-yttrium from the ⁸⁸Y-DO3A-P^{BnNH2} complex and binding to plasma proteins (predominantly to complex forming transferrin, a protein which prefers to form complexes with trivalent elements such as Fe³⁺, Co³⁺ but also Y³⁺) was shown only for <2% of the total activity administered to human plasma.

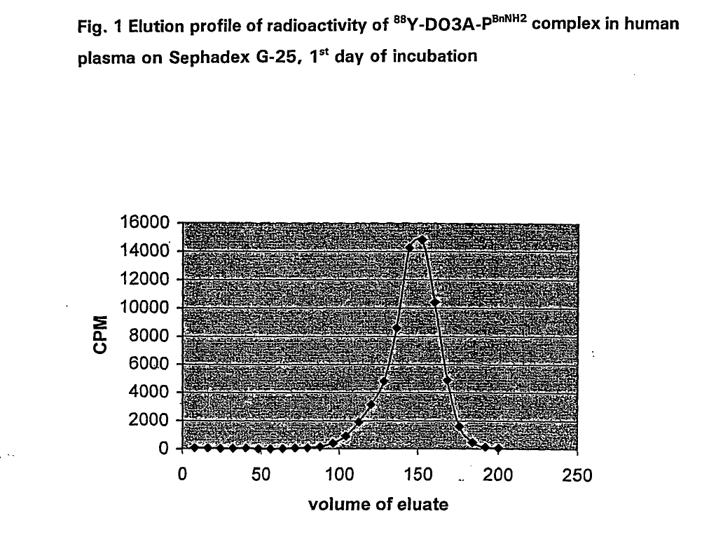

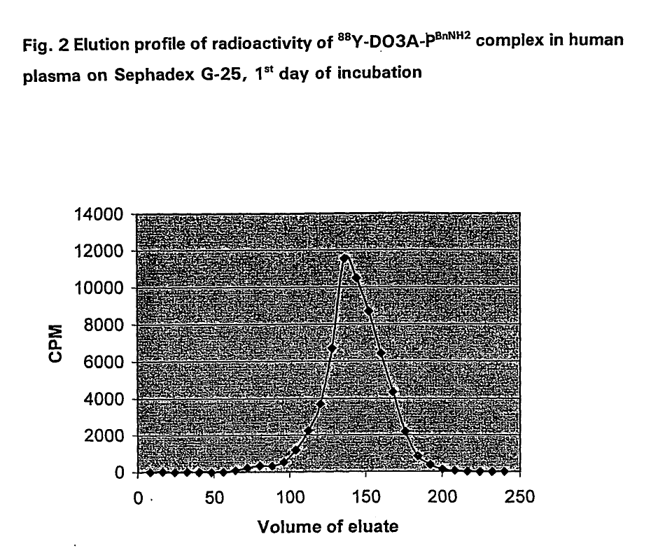

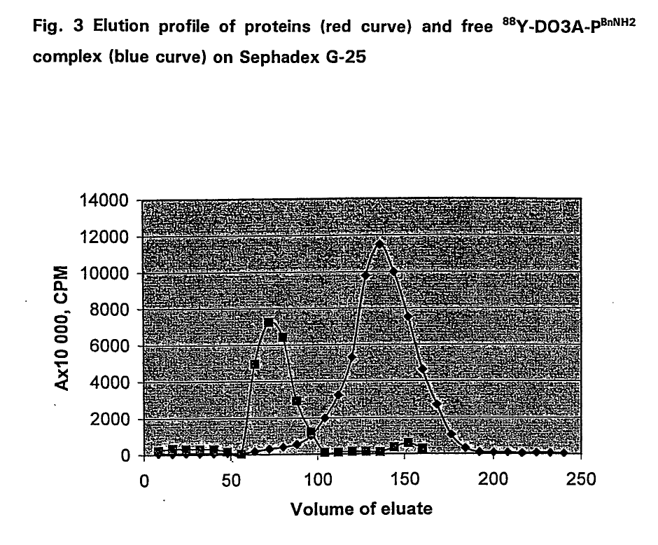

(Examples of column chromatography using Sephadex G 25 are shown in FIGS. 1, 2 and 3).

### SUMMARY

Stability, biodistribution and elimination studies in Wistar SPF rats have revealed very good biological and biochemically characteristics of Yttrium-DO3A-P^{BnNH2} complex with respect to its intended use as component of a bifunctional chelate suited for labelling of macromolecular organic substances such as polysaccharides, proteins, peptides as well as monoclonal antibodies or its fragments using suited radionuclides such as ⁹⁰Y, ⁶⁴Cu, ⁶⁷Cu, ⁶⁷Ga, ¹¹¹In, ¹⁵³Sm, ¹⁶⁶Ho, ¹⁷⁷Lu, ²⁰¹Tl, ²¹²Bi and combinations thereof. ⁸⁸Y-DO₃A-P^{BnNH2} conjugates may therefore be used advantageously as radiodiagnostic, radiotherapeutic and especially radioimmunotherapeutic agents whereas Gd-DO3A-P is especially suited as diagnostic agent for MRI.

As shown, ⁸⁸Y-DO3A-P^{BnNH2} complex is eliminated from blood, other organs and biological tissues within a short time only. It is mainly excreted over the kidneys (app. 85% activity is found after 24 h in urine compared to 4.5% activity, mean value, found in faeces).

No critical organ or tissue accumulating radioactivity was detected in the animal model used.

In case of a dissociation of the ⁸⁸Y-DO3A-P^{BnNH2} from the radioconjugate, for example a monoclonal antibody, within an organism, administered activity will be excreted within a short time from the organism by the kidneys. In addition, a high stability of the 88Y-DO3A-P^{BnNH2} complex in human plasma was shown using incubation assays following standardized conditions.

### Example 42

Conjugation Reaction of DO3A-P^{BnNCS} with MAb.

MAb BW 250/183 dissolved in phosphate buffered saline (PBS: 10 mM sodium phosphate and 150 mM sodium chloride, pH 7.2) at a concentration of 10 mg MAb/ml was adjusted to pH 8.6 by adding a 50 mM sodium borate solution dropwise. To this solution, a fourfold molar excess of DO3A-P^{BnNCS} was added as dry substance or dissolved in 1-2 ml of 50 mM sodium borate solution, pH 8.6.

After mixing, the solution was incubated at room temperature for 8 h. Free DO3A-P^{BnNCS} and other non reactive low molecular weight compounds are removed from the high molecular weight immunoconjugate and transferred to physiological saline (0.9% sodium chloride) using standard methods such as sizing gel permeation chromatography or ultrafiltration or centricon 30 spin filtration or dialysis.

Thereafter, the solution is diluted to a MAb concentration of 2 mg MAb/ml. Analytical samples were taken to determine immunoreactivity (modified Lindmo assay) and homogeneity of the immunoconjugate (SDS-PAGE, TSK 3000 gel permeation chromatography), sterilised using 0.2 μm filtration, aliquoted in sterile 5 ml glass vials, covered with sterile nitrogen and closed with sterile neoprene caps. Samples are stored at 4° C. until further use.

### Example 43

Synthesis of t-BU₃DO3A-P(O)(OMe)₂

0.4 g (0.778 mmol) of tri-t-butylester of DO3A (t-Bu₃DO3A), HP(O)(OMe)₂ (0.72 ml, 19 mmol) and 0.80 g (12 equiv.) of 30% aqueous formaldehyde were dissolved in MeOH (8 ml) and i-Pr₂NEt was added drop wise until a pH of 9-10 was reached. The solution was heated at 80° C. for 21 h. Volatiles were evaporated in vacuum and the residue was purified by column chromatography (Al₂O₃, CH₂Cl₂/MeOH/iPr₂NEt=30/6/2). Fraction containing pure ester were collected and evaporated to give pale yellow oil (1.13 g, 91%).

³¹P NMR (CDCl₃): 30.4 ppm; ESI/MS: 637.4 (M+H⁺)

### Example 44

Synthesis DO3A-P (1)

The ester from Example 43 (0.5 g) was dissolved in EtOH (10 ml) and conc. aqueous HCl was added (10 ml). The mixture was refluxed overnight. Solvents were evaporated in vacuum and the residue was purified and isolated as given in Example 1. Physical data were identical with data from Example 1.

### Example 45

Synthesis of Monomethyl Ester of DO3A-P (DO3A-P^{OMe})

The ester from Example 43 (0.5 g) was dissolved in 5 ml of 60% aqueous pyridine and heated at 50° C. for 30 h. ³¹P NMR spectrum of reaction mixture showed only a signal of product at 20.9 ppm. Purification as in Example 43 gave pale yellow oil of pure product. Yield 0.42 g (85%). ESI/MS: 623.3 (M+H⁺) 624.9 (M+Na⁺)

### Example 46

Synthesis of DO3A-P (1)

DO3A (1.0 g, 2.88 mmol), HP(O)(OMe)₂ (3.3 mg, 30 mmol) and 3 ml (30 mmol) of 30% aqueous formaldehyde were dissolved in MeOH (10 ml) and pH was adjusted to approx. 9 by addition of i-Pr₂NEt. The solution was heated at 80° C. for 24 h. Volatiles were removed in vacuum and the residue was dissolved in azeotropic HCl (20 ml) and the solution was refluxed overnight. The solution was evaporated in vacuum and the residue was purified and isolated as described in Example 1 to give the identical product.

### Example 47

Synthesis of DO3A-P^{BnNO2} (10)

1.0 g (1.94 mmol) of t-Bu₃DO3A, HP(O)(OMe)(CH₂C₆H₄NO₂) (3.34 g, 8 mmol) and 1.8 ml (10 mmol) of 30% aqueous formaldehyde were dissolved in MeOH (10 ml) and i-Pr₂NEt was added drop wise until a pH-value of approx. 9 was reached. The solution was heated at 80° C. for 24 h. Volatiles were evaporated in vacuum and the residue was purified by column chromatography (Al₂O₃, CH₂Cl₂/MeOH/iPr₂NEt=301612). Fractions containing pure ester were collected and evaporated to give yellow oil. It was dissolved in EtOH (10 ml) and azeotropic HCl (10 ml) and the solution was refluxed overnight. Solution was evaporated in vacuum and the residue was purified and isolated as described in Example 9 to give the identical product.

### Example 48

Synthesis of t-Bu₃DO3A-P(O)(OMe)(CH₂C₆H₄NH₂)

Ester from Example 47 (1.2 g, 1.6 mmol) was dissolved in EtOH (20 ml) and 10% Pd/C (0.5 g) was added. The mixture was hydrogenated (atmospheric pressure) for 48 h. Catalyst was removed by filtration and the EtOH was evaporated to give a quantitative yield of product.

³¹P NMR (CDCl₃): 36.5 ppm; ESI/MS 713.1 (M+H⁺)

### Example 49

Synthesis of t-Bu₃DO3A-P^{BnNH2}

Ester from Example 48 (1.1 g, 1.55 mmol) was dissolved in 10 ml of 60% aqueous pyridine. The solution was heated at 50° C. for 30 h. Volatiles were removed in vacuum to give quantitative yield of product as pyridinium salt.

³¹P NMR (CDCl₃): 33.2 ppm; ESI/MS 698.1 (M+H⁺).

### Example 50

Synthesis of DO3A-P(O)(OH)(CH₂C₆H₄NHC(O)CH₂Br) (DO3A-P^{BnNHAcBr})

Ester from Example 49 (1.0 g, 1.43 mmol) was dissolved in THF. iPr₂NEt (0.28 g, 1.5 mmol) was added and the solution was cooled to −10° C. Bromoacetyl bromide (0.43 g, 1.5 mmol) was dropped slowly into the solution while stirring and cooling. Amine hydrobromide was removed by filtration, solvent was evaporated in vacuum and the residue was dissolved in 50% CF₃COOH/CH₂Cl₂ (20 ml). The solution was stirred overnight. Afterwards, it was evaporated in vacuum. The residue was dissolved in 20 ml of acidified water (HCl, pH=1) and extracted with CHCl₃ to remove any remaining bromoacetic acid. The aqueous solution was cooled to −20° C. and stored at this temperature. The product was sufficiently pure for conjugation reactions.

³¹P NMR (H₂O): 30.5 ppm; ESI/MS: 651.7 (M+H⁺)

### Example 51

GdCl₃.6H₂O (g, 0.0472 mmol) was added to aqueous solution of compound 11 (50 mg in 800 mg of H₂O and 100 mg of D₂O) and pH was slowly increased to 5.5 by addition of solid KOH. Solution was stirred for 1 h at room temperature and pH was set to approx. pH 7 by careful addition of solid KOH. Thus prepared solution as well as other solutions of different concentration which were prepared by a similar approach (all containing known amount of water and gadolinium(III)) were used for relaxation measurements. The solutions gave relaxivity 7.86 mmol⁻¹ s⁻¹ (at 10 MHz). Exchange half-life of coordinated water molecule 14 ns was determined (from temperature dependence of 170 NMR parameters).

### Example 52

Solution of gadolinium(III) complex of compound 1 for relaxation measurements were prepared similarly to Example 51. The solutions gave relaxivity 7.54 mmol-1 s⁻¹ (at 10 MHz). Exchange half-life of coordinated water molecule 70 ns was determined (from temperature dependence of ¹⁷O NMR parameters).

### Example 53

Synthesis of Triethyl Ester of DO3A (Et3DO3A)

Cyclen (5 g, 29 mmol) was dissolved in dry CH₂Cl₂ (500 ml) and BrCH₂COOEt (13.23 g, 2.73 equiv.) dissolved in 50 ml dry CH₂Cl₂ was slowly added during 14 h with efficient stirring. After 24 h of stirring white precipitate was filtered off and filtrate was evaporated in vacuum to thick oil. It was diluted with 2 ml of CH₂Cl₂ and left crystallized overnight. The crystalline solid was filtered, washed with a small amount of CH₂Cl₂ and Et₂O and left to dry on air. Yield of Et₃DO3A.2HBr was 6.53 g (38%).

Elementary analysis (calc.): C 37.63 (40.55) H 6.44 (6.81) N 8.78 (9.46) Br 25.34 (26.98)

ESI/MS: 431.3 (M+H⁺)

¹H NMR (D20): 1.15 ppm (t, 6H, ²J(HH)=7.1 Hz), 1.20 ppm (t, 3H, ²J(HH)=7.1 Hz), 2.80-3.34 ppm (several broad m, 14H, ring protons), 3.48-3.63 ppm (bm, 8H, ring plus NCH₂C protons), 4.09-4.03 ppm (several m, 6H, ester CH₂);

¹³C NMR (D20): 15.83 and 15.90 ppm (2×CH₃), 44.73+50.14+51.84+54.75+55.56+56.83 ppm (ring carbon atoms and ester CH₂), 64.80 and 66.17 ppm (NCH₂), 166.69 and 175.48 ppm (COOH)

### Example 54

Synthesis of DO3A-P^{BnNHAcBr}

DO3A-P^{BnNH2} (0.5 g, 0.94 mmol) was dissolved in 10 ml of water and iPr₂NEt (1.82 g, 15 equiv.) was added. Bromoacetyl bromide (2.85 g, 15 equiv.) was dissolved in 10 ml of CHCl₃ and both solutions were mixed and intensively stirred. After 1 h, the same amount of iPr₂NEt was added to the two-phase mixture followed by the same amount of the bromide in 5 ml of CHCl₃. The mixture was stirred for 1 additional hour. Two phases were separated and aqueous phase was washed with 2×10 ml of CHCl₃. Aqueous phase was acidified with diluted HCl to pH 1 and extracted ten times with 10 ml of CHCl₃. Aqueous phase was decolourised with charcoal and evaporated to oil (at bath temperature 30° C.). The oil was diluted with 2 ml of water and the solution was characterized and finally stored at −20° C. Aliquots of the solution may be directly used for conjugation reactions. Data were identical with Example 50.

### Example 55

Synthesis of DO3A-P by Oxidation of DO3A-P^{H}

A sample of hydrochloride of DO3A-P^{H} (1.5 g, approx. 2.8 mmol) was dissolved in 10 ml of water. 1.2 equivalents of bromine (in form of bromine water) were added drop wise—next drop was added after decolourising of the reaction mixture. Solvent was removed in vacuum and the residue was purified on ion exchange resins as described in Example 1 to obtain an identical product. The yield amounted to 1.18 g of product trihydrate (85%).

TABLE 1
Biodistribution of ⁸⁸Y-DO3A-P^{BnNH2} complex in Wistar SPF rats
(percent dose in whole organ)

| | 5 min | | 60 min | | 120 min | | 24 h | |
|---|---|---|---|---|---|---|---|---|
| Liver | 1.79 | 0.23 | 0.3 | 0.07 | 0.15 | 0.11 | 0.1 | 0.01 |
| Adrenals | 0.03 | 0.01 | 0.01 | 0.01 | 0.01 | 0.01 | 0.01 | 0 |
| Kidney | 15.14 | 5.66 | 1.47 | 0.44 | 1.11 | 0.93 | 1.06 | 0.48 |
| Lung | 1.01 | 0.5 | 1.14 | 0.05 | 0.03 | 0.02 | 0.01 | 0 |
| Heart | 0.35 | 0.05 | 0.03 | 0.01 | 0.01 | 0.01 | 0.01 | 0 |
| Spleen | 0.15 | 0.03 | 0.02 | 0.01 | 0.02 | 0.01 | 0.01 | 0 |
| Stomach | 0.61 | 0.1 | 0.08 | 0.01 | 0.23 | 0.38 | 0.04 | 0.06 |
| Intestine | 2 | 0.1 | 0.88 | 0.07 | 3.48 | 5.51 | 0.2 | 0.29 |
| Colon | 1.22 | 0.08 | 0.14 | 0.03 | 0.16 | 0.22 | 2.52 | 1.42 |
| Testes | 0.41 | 0.03 | 1.11 | 0.03 | 0.03 | 0.02 | 0.01 | 0 |
| Thyroid | 0.07 | 0 | 0.01 | 0.01 | 0.01 | 0.01 | 0.01 | 0 |
| Brain | 0.08 | 0.02 | 0.02 | 0.01 | 0.01 | 0.01 | 0.01 | 0 |
| Femur | 0.1 | 0.1 | 0.02 | 0.01 | 0.01 | 0.01 | 0.01 | 0 |

TABLE 2
Biodistribution of ⁸⁸Y-DO3A-P^{BnNH2} complex in Wistar SPF rats
(percent dose per 1 g of organ)

| | 5 min | | 60 min | | 120 min | 24 h |
|---|---|---|---|---|---|---|
| Blood | 1.03 | 0.13 | 0.09 | 0.03 | 0.02 ± 0.01 | 0.004 ± 0.003 |
| Plasma | 2.08 | 0.25 | 0.17 | 0.05 | 0.02 ± 0.02 | 0.005 ± 0.003 |
| Pancreas | 0.34 | 0.03 | 0.05 | 0.01 | 0.02 ± 0.01 | 0.01 ± 0 |
| Liver | 0.25 | 0.03 | 0.04 | 0.01 | 0.02 ± 0.01 | 0.02 ± 0 |
| Adrenals | 0.48 | 0.22 | 0.15 | 0.14 | 0.21 ± 0.16 | 0.1 ± 0.03 |
| Kidney | 8.52 | 3.38 | 0.76 | 0.27 | 0.59 ± 0.47 | 0.62 ± 0.25 |
| Lung | 0.73 | 0.13 | 0.09 | 0.02 | 0.02 ± 0.02 | 0.01 ± 0 |
| Heart | 0.45 | 0.07 | 0.04 | 0.01 | 0.01 ± 0.01 | 0.01 ± 0.01 |
| Spleen | 0.29 | 0.08 | 0.04 | 0.01 | 0.04 ± 0.03 | 0.03 ± 0 |
| Stomach | 0.26 | 0.05 | 0.04 | 0 | 0.08 ± 0.13 | 0.01 ± 0.01 |
| Intestine | 0.28 | 0.03 | 0.11 | 0.03 | 0.53 ± 0.83 | 0.03 ± 0.05 |
| Colon | 0.17 | 0.05 | 0.02 | 0.01 | 0.02 ± 0.03 | 0.44 ± 0.26 |
| Testes | 0.14 | 0.01 | 0.04 | 0.01 | 0.01 ± 0.01 | 0 ± 0 |
| Skin | 0.46 | 0.04 | 0.09 | 0.02 | 0.03 ± 0.03 | 0.02 ± 0.02 |
| Muscle | 0.22 | 0.03 | 0.03 | 0.01 | 0 ± 0.01 | 0.006 ± 0.002 |
| Thyroid | 0.86 | 0.11 | 0.12 | 0.08 | 0.1 ± 0.16 | 0.06 ± 0.04 |
| Brain | 0.04 | 0.01 | 0.01 | 0 | 0 ± 0.01 | 0 ± 0 |
| Fat | 0.32 | 0.06 | 0.07 | 0.02 | 0.02 ± 0.06 | 0.05 ± 0.05 |
| Femur | 0.21 | 0.03 | 0.04 | 0.02 | 0.02 ± 0.02 | 0.02 ± 0.01 |

TABLE 3
Biodistribution of ⁸⁸Y-DO3A-P^{BnNH2} complex in Wistar SPF rats
(percent dose per 1% body weight)[D

| | 5 min | 60 min | 120 min | 24 h |
|---|---|---|---|---|
| Blood | 2.28 ± 0.33 | 0.19 ± 0.06 | 0.03 ± 0.03 | 0.009 ± 0.007 |
| Plasma | 4.61 ± 0.56 | 0.37 ± 0.07 | 0.06 ± 0.05 | 0.01 ± 0.007 |
| Pancreas | 0.76 ± 0.07 | 0.1 ± 0.02 | 0.04 ± 0.03 | 0.03 ± 0.01 |
| Liver | 0.56 ± 0.08 | 0.09 ± 0.01 | 0.05 ± 0.03 | 0.03 ± 0.01 |
| Adrenals | 1.05 ± 0.45 | 0.35 ± 0.35 | 0.48 ± 0.35 | 0.21 ± 0.06 |
| Kidney | 18.7 ± 6.92 | 1.64 ± 0.42 | 1.33 ± 1.08 | 1.32 ± 0.56 |
| Lung | 1.61 ± 0.32 | 0.19 ± 0.04 | 0.05 ± 0.03 | 0.02 ± 0.01 |
| Heart | 1 ± 0.15 | 0.09 ± 0.03 | 0.03 ± 0.03 | 0.02 ± 0.01 |
| Spleen | 0.65 ± 0.14 | 0.1 ± 0.03 | 0.08 ± 0.06 | 0.06 ± 0.01 |
| Stomach | 0.58 ± 0.13 | 0.08 ± 0.01 | 0.18 ± 0.29 | 0.03 ± 0.03 |
| Intestine | 0.63 ± 0.09 | 0.26 ± 0.04 | 1.14 ± 1.77 | 0.07 ± 0.1 |
| Colon | 0.37 ± 0.09 | 0.04 ± 0.01 | 0.05 ± 0.06 | 0.93 ± 0.58 |
| Testes | 0.32 ± 0.03 | 0.09 ± 0.02 | 0.03 ± 0.02 | 0.01 ± 0 |
| Skin | 1.02 ± 0.04 | 0.2 ± 0.02 | 0.06 ± 0.06 | 0.03 ± 0.03 |
| Muscle | 0.5 ± 0.08 | 0.06 ± 0.02 | 0.01 ± 0.02 | 0.012 ± 0.005 |
| Thyroid | 1.91 ± 0.2 | 0.28 ± 0.22 | 0.22 ± 0.36 | 0.12 ± 0.09 |
| Brain | 0.09 ± 0.03 | 0.02 ± 0.01 | 0.01 ± 0.01 | 0.01 ± 0.01 |
| Fat | 0.72 ± 0.15 | 0.16 ± 0.06 | 0.05 ± 0.13 | 0.11 ± 0.1 |
| Femur | 0.47 ± 0.06 | 0.09 ± 0.04 | 0.05 ± 0.05 | 0.04 ± 0.02 |

TABLE 4
Formation of ⁹⁰Y-DO3A-P^{BnNH2}-complex. Effect of reaction time and
temperature on radiochemical yield

Testing conditions: [Y] = 1,2 · 10⁻³ mol/l; ratio of ligand: Y = 1:1;
pH = 5.5;
reaction temperature: 25° C. and 37° C.

| | radiochemical | yield (%) |
|---|---|---|
| reaction time (min) | 25° C. | 37° C. |
| 0 | 88 | 88 |
| 15 | 97 | 98 |
| 30 | 97 | 98 |
| 45 | 97 | 98 |
| 60 | 97 | 97 |
| 90 | 98 | 98 |
| 120 | 98 | 99 |

TABLE 5
Formation of ⁹⁰Y-DO3A-P^{BnNH2}-complex. Effect of pH on
radiochemical yield

Testing conditions: [Y] = 1.5 · 10⁻⁵ mol/l; ratio of ligand: Y = 3:1;
reaction time: 60 min, reaction temperature: 25° C.

| pH | radiochemical yield (%) |
|---|---|
| 2.0 | 12 |
| 3.0 | 54 |
| 3.9 | 77 |
| 4.4 | 93 |
| 4.9 | 97 |
| 5.6 | 95 |
| 6.0 | 93 |
| 6.2 | 97 |
| 6.6 | 98 |
| 6.8 | 98 |
| 8.0 | 98 |
| 8.9 | 91 |

TABLE 6
Formation of ⁹⁰Y-DO3A-P^{BnNH2}-complex. Effect of ligand
concentration on radiochemical yield

Testing conditions: [Y] = 1.2 · 10⁻³ mol/l;
[ligand] = 1.2 · 10⁻³ mol/l to 8.4 · 10⁻³ mol/l;
pH = 5.2; reaction time: 60 min; reaction temperature: 25° C.

| ratio of ligand:Y | radiochemical yield (%) |
|---|---|
| 1:1 | 94 |
| 2:1 | 97 |
| 3:1 | 98 |
| 4:1 | 99 |
| 5:1 | 97 |
| 6:1 | 99 |
| 7:1 | 97 |

TABLE 7
Cumulative excretion of radioactivity after administration of ⁸⁸Y-
DO3A-P^{BnNH2} complex to Wistar SPF rats

| Interval | Urine | Faeces |
|---|---|---|
| 0-2 h | 79.4 ± 5.2% | — |
| 0-24 h | 84.3 ± 5.0% | 3.6 ± 2.8% |

TABLE 8
Stability of ⁸⁸Y-DO3A-P^{BnNH2} complex in human plasma

| | Experiment No. 1 | | Experiment No. 2 | |
|---|---|---|---|---|
| | Low | High | Low | High |
| | molecular | molecular | molecular | molecular |
| Interval | weight form | weight form | weight form | weight form |

| | | | | |
|---|---|---|---|---|
| Day 0 | 99.72% | 0.28% | 99.90% | 0.10% |
| Day 3 | 99.25% | 0.75% | 99.44% | 0.56% |
| Day 5 | 99.28% | 0.72% | 99.09% | 0.91% |
| Day 7 | 99.21% | 0.79% | 99.27% | 0.73% |
| Day 10 | 99.09% | 0.91% | 98.92% | 1.08% |
| Day 12 | 98.83% | 1.17% | 98.65% | 1.35% |
| Day 14 | 98.32% | 1.68% | 98.20% | 1.80% |

1. A compound of formula 1, 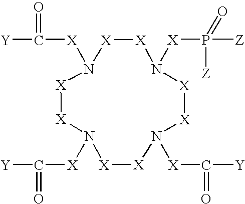
wherein
each X is independently selected from C(R¹)₂₁ CH₂, CHR¹, CHR² or CR¹R²,
each Z is independently R¹, R², —OR¹ or OR²,
Y is independently OH, OM, OR¹, OR², NH₂, NHR¹, NHR₂, NR¹R², N(R¹)₂ or N(R²)₂ and M is a cation,
each R¹ is independently selected from an organic radical having from 1-20 carbon atoms, and
each R² is independently selected from a functional group or an organic radical having from 1-20 carbon atoms carrying at least one functional group,
or an optical isomer, a coordination compound or a salt thereof.

2. The compound of claim 1, wherein the functional group is selected from OR¹, Cl, Br, I, NO₂, N(R¹)₂, COOR¹, NCS, NHCOCH₂Br, wherein R¹ is defined as in claim 1.

3. The compound of claim 1 or 2, wherein X is CH₂.

4. The compound of claims 1-3, wherein Z is C₁₋₆ alkyl, C₁-C₃ alkoxy, —Oₙ—C₁-C₂ alkyl-aryl or —Oₙ-aryl, wherein n is 0 or 1.

5. The compound of claims 1-3, wherein Z is —Oₙ—(CH₂)₁₋₆-Q, —Oₙ—(CH₂)₁₋₄—Ph-Q or —O—Ph-Q, wherein Q is —NH₂, —COOH, —NCS or —NHCOCH₂Br and n is 0 or 1.

6. A metal complex of a compound of any one of claims 1-5.

7. The complex of claim 6, wherein the metal is a radioisotope.

8. The complex of claim 7, wherein the radioisotope is selected from ⁶⁴Cu, ⁶⁷Cu, ⁶⁷Ga, ⁹⁰Y, ¹¹¹In, ¹⁵³Sm, ¹⁶⁶Ho, ¹⁷⁷Lu, ²⁰¹Tl, ²¹²Bi and combinations thereof.

9. The complex of claim 6, wherein the metal is Gd.

10. A conjugate of a compound with a biomolecule wherein the coupling to said biomolecule is formed through P-alkyl or P—O-alkyl and wherein the compound is of the formula II 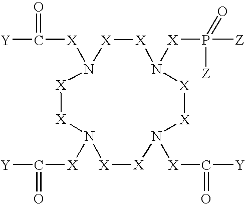
wherein
each X is independently selected from C(R¹)₂ or CR¹R²,
each Z is independently OH, R¹, R², —OR¹ or OR² or OM and M is a cation,
Y is independently OH, OM, OR¹, OR², NR¹R², N(R¹)₂ or N(R²)₂ and M is a cation,
each R¹ is independently selected from H or an organic radical having from 1-20 carbon atoms, and each R² is independently selected from a functional group or an organic radical having from 1-20 carbon atoms carrying at least one functional group,
or an optical isomer, a coordination compound or a salt thereof, or a metal complex thereof.

11. The conjugate of claim 10, wherein the biomolecule is selected from peptides, proteins, glycoproteins, oligo- and polysaccharides, oligo- and polyaminosugars and nucleic acids.

12. The conjugate of claim 11, wherein the biomolecule is an antibody or antibody fragment.

13. A pharmaceutical composition comprising a compound of any one of claims 1-5, a metal complex of any one of claims 6-9 or a conjugate of any one of claims 10-12 together with pharmaceutically acceptable carriers, diluents or adjuvants.

14. The composition of claim 13 for diagnostic applications.

15. The composition of claim 14 for radioimaging.

16. The composition of claim 14 for magnetic resonance imaging.

17. The composition of claim 13 for therapeutic applications.

18. The composition of claim 17 for radiotherapy.

19. The composition of claim 17 for neutron capture therapy.

20. A method of administering a subject in need thereof a diagnostically or therapeutically effective amount of a compound of any one of claims 1-5, a metal complex of any one of claims 6-9 or a conjugate of any one of claims 10-12 together with pharmaceutically acceptable carriers, diluents or adjuvants.
